(19) United States
(12) Patent Application Publication
(10) Pub. No.: US 2023/0112662 A1
(43) Pub. Date: Apr. 13, 2023

(54) METHODS AND COMPOSITIONS FOR REPAIRING THE BLOOD-BRAIN BARRIER AND OTHER ENDOTHELIUM

(71) Applicant: University Hospitals Cleveland Medical Center, Cleveland, OH (US)

(72) Inventors: Andrew A. Pieper, Cleveland, OH (US); Min-Kyoo Shin, Cleveland, OH (US); Edwin Vázquez-Rosa, Cleveland, OH (US); Kalyani Chaubey, Cleveland, OH (US); Matasha Dhar, Cleveland, OH (US)

(21) Appl. No.: 17/497,850

(22) Filed: Oct. 8, 2021

(51) Int. Cl. A61K 31/4045; A61P 25/28

(52) U.S. Cl. CPC A61K 31/4045; A61P 25/28

(57) ABSTRACT

Provided herein, in one aspect, is a method of treating a disease or condition associated with insufficient levels of claudin-5, comprising providing a subject having insufficient levels of claudin-5, and administering an effective amount of a P7C3 compound to the subject.

### CROSS REFERENCE TO RELATED APPLICATIONS

This application claims priority to and the benefit of U.S. Provisional Patent Application No. 63/090,160 filed Oct. 9, 2020, which is incorporated herein by reference in its entirety.

### FIELD

The present disclosure relates in general to methods and compositions for repairing damaged endothelial cells such as the blood-brain barrier.

### BACKGROUND

Traumatic brain injury (TBI) is commonly caused by motor vehicle accidents, falls, contact sports, explosions, or assaults, with an estimated ˜70 million people worldwide sustaining a TBI every year (1). In the United States alone, there are almost 3 million annual emergency department visits for TBI treatment (2) and ˜5 million people living with TBI-related disabilities, translating to an annual cost of ˜$80 billion (3-5). Many TBI survivors experience chronic diffuse axonal degradation and nerve cell death (6-8) associated with sensorimotor impairment, cognitive dysfunction, and emotional dysregulation, as well as increased risk of developing Alzheimer's disease (AD), Parkinson's disease (PD), vascular dementia, and chronic traumatic encephalopathy (CTE) (9,10). endothelium

The initial clinical management of TBI is confined to acute measures such as reducing intracranial pressure and edema (11) while maintaining oxygenation and meeting metabolic demand of the injured brain (12,13). For survivors, the only chronic treatment options are prolonged physical and cognitive rehabilitation accompanied by symptom-driven medication. Unfortunately, such approaches rarely slow the long-term deterioration in neurologic function (14). In essence, TBI produces a chronic pathology that is triggered by injury-initiated persistent neurodegeneration and leads to life-long detrimental effects on multiple health outcomes (6,15).

Despite decades of research, there remains a tremendous unmet need for new disease-modifying therapies that can mitigate post-TBI chronic neurodegeneration (16).

### SUMMARY

Provided herein, in one aspect, is a method of treating a disease or condition associated with insufficient levels of claudin-5, comprising providing a subject having insufficient levels of claudin-5, and administering an effective amount of a P7C3 compound to the subject.

In some embodiments, the subject has a decreased level of claudin-5 protein compared to a healthy subject. In some embodiments, the subject has a decreased level of claudin-5 mRNA compared to a healthy subject. In some embodiments, the subject has a decreased level of functional claudin-5 protein compared to a healthy subject. In some embodiments, the subject has a decreased level of post-translational modification (e.g., phosphorylation) of claudin-5 protein compared to a healthy subject.

In some embodiments, the subject has damaged or deteriorated endothelial cells located in the brain, kidney, lung, heart, cornea, or digestive tissues. In some embodiments, upon administration, the P7C3 compound repairs the damaged or deteriorated endothelial cells.

In some embodiments, the subject has a deteriorated, damaged or impaired blood-brain barrier (BBB). In some embodiments, the subject shows brain permeability due to impaired BBB. In some embodiments, upon administration, the P7C3 compound restores integrity, structure and/or function of the subject's BBB.

In some embodiments, the P7C3 compound comprises 3,6-dibromo-3-fluoro-N-(3-methoxyphenyl)-9H-carbazole-9-propanamine.

In some embodiments, the disease or condition is selected from Stroke, Traumatic Brain Injury, Velocardial Facial Syndrome, Epilepsy, Alzheimer's disease (AD), Glioblastoma, Multiple sclerosis, Bipolar Disorder, Obsessive Compulsive Disorder, ADHD (attention-deficit/hyperactivity disorder), Depression, Pain Disorders, Schizophrenia, and Heart Failure.

In some embodiments, the disease or condition is selected from subarachnoid hemorrhage, schizophrenia, major depression, bipolar disorder, normal aging, epilepsy, traumatic brain injury and/or a visual symptom associated therewith, post-traumatic stress disorder, Parkinson's disease, Alzheimer's disease, Down syndrome, spinocerebellar ataxia, amyotrophic lateral sclerosis, Huntington's disease, stroke, radiation therapy, chronic stress, abuse of a neuro-active drug, retinal degeneration, spinal cord injury, peripheral nerve injury, physiological weight loss associated with various conditions, cognitive decline and/or general frailty associated with normal aging and/or chemotherapy, chemotherapy induced neuropathy, concussive injury, crush injury, peripheral neuropathy, diabetic neuropathy, post-traumatic headache, multiple sclerosis, retinal degeneration and dystrophy (such as Leber congenital amaurosis, retinitis pigmentosa, cone-rod dystrophy, microphthalmia, anophthalmia, myopia, and hyperopia), spinal cord injury, traumatic spinal cord injury, peripheral nerve injury (such as peripheral nerve crush injury, neonatal brachial plexus palsy, and traumatic facial nerve palsy), retinal neuronal death related diseases (such as glaucoma and age related macular degeneration, diabetic retinopathy, retinal blood vessel occlusions, retinal medication toxicity (such as what amino glycosides or plaquenil can cause), retinal trauma (e.g., post-surgical), retinal infections, choroidal dystrophies, retinal pigmentary retinopathies, inflammatory and cancer mediated auto immune diseases that result in retinal neuronal cell death), Autism, Stargardt disease, Kearns-Sayre syndrome, Pure neurosensory deafness, Hereditary hearing loss with retinal diseases, Hereditary hearing loss with system atrophies of the nervous system, Progressive spinal muscular atrophy, Progressive bulbar palsy, Primary lateral sclerosis, Hereditary forms of progressive muscular atrophy and spastic paraplegia, Frontotemporal dementia, Dementia with Lewy bodies, Corticobasal degeneration, Progressive supranuclear palsy, Prion disorders causing neurodegeneration, Multiple system atrophy (olivopontocerebellar atrophy), Hereditary spastic paraparesis, Friedreich ataxia, Non-Friedreich ataxia, Spinocerebellar atrophies, Amyloidoses, Metabolic-related (e.g., Diabetes) neurodegenerative disorders, Toxin-related neurodegenerative disorders, Multiple sclerosis, Charcot Marie Tooth, Diabetic neuropathy, Metabolic neuropathies, Endocrine neuropathies, Orthostatic hypotension, Creutzfeldt-Jacob Disease, Primary progressive aphasia, Frontotemporal Lobar Degeneration, Cortical blindness, Shy-Drager Syndrome (Multiple, System Atrophy with Orthostatic Hypotension), Diffuse cerebral cortical atrophy of non-Alzheimer type, Lewy-body dementia, Pick disease (lobar atrophy), Thalamic degeneration, Mesolimbocortical dementia of non-Alzheimer type, Nonhuntingtonian types of chorea and dementia, Cortical-striatal-spinal degeneration, Dementia-Parkinson-amyotrophic lateral sclerosis complex, Cerebrocerebellar degeneration, Cortico-basal ganglionic degeneration, Familial dementia with spastic paraparesis or myoclonus, and Tourette syndrome.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1C. P7C3-A20 restores cognition in chronic TBI. (FIG. 1A) Experimental schematic. (FIG. 1B) All groups showed equal learning at 15 months. At 19 months, TBI-Veh exhibited learning deficits on day 4 compared to Sham-Veh. (FIG. 1C) Platform crossings in the memory probe test show expected aging-related decline in Sham-Veh mice, and TBI-Veh showed significant impairment relative to Sham-Veh. Memory function was fully restored in TBI-P7C3-A20 at both time points. Values are mean±SEM. Individual data points represent individual animals. Significance determined by repeated-measure two-way ANOVA for learning, one-way ANOVA for memory and Dunnett's post hoc analysis, * p<0.05, ** p<0.01, *** p<0.001, relative to TBI-Veh.

FIGS. 2A-2H. P7C3-A20 restores the BBB and arrests neurodegeneration in chronic TBI. (FIG. 2A) Neuronal cell loss measured by immunostaining for NeuN in CTX and hippocampal dentate gyrus (DG) reveals reduced neurons in TBI-Veh and TBI-P7C3-A20 relative to Sham-Veh (Scale bar=200 m). (FIG. 2B) Ongoing neurodegeneration measured by silver staining of CTX, CC, fimbria, internal capsule, and hippocampus (HPC) demonstrate significant increase in TBI-Veh relative to Sham-Veh, with TBI-P7C3-A20 restored to Sham-Veh levels (Scale bar=20 m). (FIG. 2C) Transmission electron microscopy of cortex (CTX) and hippocampus (HPC) show BBB capillary endothelium breaks (red arrows) in TBI-Veh, but not in Sham-Veh or TBI-P7C3-A20. (scale bar=0.5 m and 1 m). (FIG. 2D) Peripheral IgG infiltration into the brain, and neuroinflammation via Iba1 microglial activation, are increased in CTX, corpus callosum (CC) and hippocampus (HPC) in TBI-Veh compared to Sham-Veh, and restored to Sham-Veh levels in TBI-P7C3-A20 (scale bar=20 m). (FIG. 2E) Quantification of NeuN. (FIG. 2F) Quantification of silver staining. (FIG. 2G) Quantification of BBB capillary endothelium breaks was determined by number of capillary EC breaks per an average of 113 capillaries. (FIG. 2H) Quantification of IgG infiltration and Iba1 microglial activation. All values are mean±SEM. Individual data points represent individual animals. Significance determined by one-way ANOVA and Dunnett's post hoc analysis. * p<0.05, ** p<0.01, *** p<0.001 relative to TBI-Veh.

FIGS. 3A-3H P7C3-A20 restores BBB capillary endothelium length and tight junction protein expression in chronic TBI, and also increases basal pericyte abundance. (FIGS. 3A-3F) CD31 staining was used to determine capillary endothelium length, and PDGFRβ staining was used to quantify pericytes. Representative pictures (FIGS. 3A,3B) and quantification (FIGS. 3C-3F) from CTX and HPC of Sham-Veh, TBI-Veh, and TBI-P7C3-A20 show reduced capillary endothelium length in TBI-Veh relative to Sham-Veh, which is restored to normal by P7C3-A20. TBI-P7C3-A20 shows an increase in the number of pericytes per capillary length in the CTX and the HPC relative to both Sham-Veh and TBI-Veh (Scale bar=25 m). (FIGS. 3G,3H) Western blot analysis of tight junction proteins reveals that TBI-P7C3-A20 animals have higher levels of claudin-5 in the CTX and HPC, and of ZO1 in the CTX. Values are mean±SEM. Individual data points represent individual animals. Significance was determined by one-way ANOVA and Dunnett's post hoc analysis, n=3; * p<0.05, ** p<0.01, *** p<0.001 relative to TBI-Veh.

FIGS. 4A-4B. P7C3-A20 protects endothelial cells in vivo and in vitro. (FIG. 4A) LPS-damage to the BBB was measured by brain permeability of fluorescent-conjugated 3 kD dextran, with greater dextran entry in LPS-Veh relative to Veh-Veh. LPS-P7C3-A20, however, showed significant reduction relative to LPS-VEH. Values are mean±SEM. Individual data points represent individual animals. Significance determined by one-way ANOVA and Tukey's post hoc analysis. * p<0.05, ** p<0.01, *** p<0.001, **** p<0.0001 relative to Veh-LPS. (FIG. 4B) Cultured human brain microvascular endothelial cells exposed to 0.1 mM H₂O₂ showed 50% reduction in cell viability, which was blocked by P7C3-A20 in a dose-response manner. Values are presented as mean±SEM. Individual data points represent individual experiments of six replicates. Significance was determined by one-way ANOVA and Dunnett's post hoc analysis, n=5; * p<0.05, ** p<0.01, *** p<0.001, **** p<0.0001 relative to H₂O₂ cells treated only.

FIGS. 5A-5D. P7C3-A20 treatment acutely protects BBB integrity after TBI. (FIG. 5A) Acute damage to the BBB was determined by brain permeability of fluorescent-conjugated 3 kD dextran at different timepoints after TBI, with significantly higher dextran permeability at 3 hours after the injury compared against Sham group, and returning to normal thereafter. Values are mean±SEM. Individual data points represent individual animals. Significance was determined by one-way ANOVA and Dunnett's post hoc analysis, * p<0.05 relative to Sham. (FIG. 5B) Experimental schematic. Efficacy of P7C3-A20 in protecting the BBB was determined at (FIG. 5C) 3 and (FIG. 5D) 6 hours after TBI. Values are mean±SEM. Individual data points represent individual animals. Significance was determined by one-way ANOVA and Dunnett's post hoc analysis, * p<0.05 relative to TBI-Veh.

FIG. 6. P7C3-A20 does not restore normal swim speed in chronic TBI. Mean swimming speed was chronically reduced after TBI at 19 months, and this deficit was not ameliorated by P7C3-A20. Values are mean±SEM. Individual data points represent individual animals. Significance was determined by one-way ANOVA and Tukey's post hoc analysis, * p<0.05, ** p<0.01 relative to Sham-Veh.

FIG. 7. Representative transmission electron microscopy images from the cerebral cortex of Sham-Veh, TBI-Veh and TBI-P7C3-A20 animals. Red arrows indicate breaks in endothelium.

FIG. 8. Representative transmission electron microscopy images from the hippocampus of Sham-Veh, TBI-Veh and TBI-P7C3-A20 animals. Red arrows indicate breaks in endothelium.

FIG. 9. Western blot analysis of tight junction proteins reveals that P7C3-A20 treatment restores expression of ZO1 and Claudin 5 in the cortex of TBI animals. Remarkably, P7C3-A20 treatment increases Claudin 5 levels in un-injured Sham animals as well. Values are mean±SEM. Significance was determined by student's t-test.

### DETAILED DESCRIPTION

Chronic neurodegeneration, a major cause of the long-term disabilities that afflict survivors of traumatic brain injury (TBI), is linked to an increased risk for late-life neurodegenerative disorders, including Alzheimer's disease, Parkinson's disease, vascular dementia, and chronic traumatic encephalopathy. Disclosed herein, among other things, is the surprising restoration of blood-brain barrier (BBB) structure and function by P7C3-A20 or the P7C3 class of compounds when administered long after infliction of TBI (e.g., up to 12 months or longer after TBI). This pharmacotherapy was associated with cessation of chronic neurodegeneration and recovery of normal cognitive function; benefits that persisted long after treatment cessation. Pharmacologic renewal of BBB integrity may thus provide a new treatment option for patients who have suffered a remote TBI, or other neurological conditions associated with BBB deterioration. Furthermore, given the surprising finding that P7C3 compounds can elevate levels of claudin-5, these compounds can also be used to repair other damaged or deteriorated endothelial cells such as kidney, lung, heart, cornea, digestive tissues, etc.

### Definitions

Unless defined otherwise, all technical and scientific terms used herein have the meaning commonly understood by one of ordinary skill in the art to which the disclosure pertains. The Definitions section at paragraphs [1001]-[1031] of U.S. Publication No. 2013/0040977 is incorporated herein by reference. Specific terminology is defined below.

As used in the specification and the appended claims, the singular forms “a,” “an,” and “the” include plural referents unless the context clearly dictates otherwise.

As used herein, the term “about” means within 20%, more preferably within 10% and most preferably within 5%.

The terms “treating” and “treatment” as used herein refer to reduction in severity and/or frequency of symptoms, elimination of symptoms and/or underlying cause, and improvement or remediation of damage.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing and/or that it is included on the Inactive Ingredient Guide prepared by the U.S. Food and Drug administration.

As used herein, the term “patient” or “individual” or “subject” refers to any person or mammalian subject for whom or which therapy is desired, and generally refers to the recipient of the therapy to be practiced according to the disclosure.

A number of small molecules with in vivo neuroprotective properties (the “P7C3 class of compounds”), including P7C3-A20 (3,6-dibromo-3-fluoro-N-(3-methoxyphenyl)-9H-carbazole-9-propanamine), have been previously identified and disclosed in U.S. Pat. No. 8,362,277; U.S. Publication No. 2011/0015217; U.S. Publication No. 2012/0022096; U.S. Publication No. 2013/0040977 and U.S. application Ser. No. 14/339,772 filed Jul. 24, 2014, all of which are hereby incorporated herein by reference in their entirety, in particular the compounds disclosed in the Examples section. It should be noted that while the Examples used P7C3-A20, a number of other P7C3 class of compounds can be used in place of or in combination with P7C3-A20 as a therapeutic agent.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### BBB and Claudin-5

The blood-brain barrier (BBB) is formed by a tightly packed monolayer of non fenestrated endothelial cells lining the brain capillaries which are enveloped by pericytes and perivascular astrocytes. The BBB forms a protective layer in the central nervous system (CNS) strictly limiting molecular exchange between the circulating blood and brain microenvironment. This is to ensure a constant state of homeostasis for efficient neural signaling. Accounting for just 2% of bodily mass, the brain and neuronal functions consume as much as 20% of the body's oxygen and glucose needs despite a lack of energy reserves in the brain. Therefore, blood vessels in the brain provide vital energy and nutrients in response to the metabolic demands of neurons (a process known as hyperaemia). Cerebral capillaries account for 85% of vessel length in the brain, providing a surface area of ˜12 m² of the endothelial surface for molecular exchange and an approximate 1.1 ratio of capillaries to neurons. Owing to the restrictive regenerative capacities of neurons, there is a vital need to limit the unrestricted movement of material between the blood and brain and vice versa to prevent unwanted neuronal excitation. The BBB is formed by a monolayer of tightly packed, specialized endothelial cells along the cerebral vasculature that are mechanically linked together by tight junction protein complexes that eliminate the paracellular space between neighboring endothelial cells. Additionally, the presence of numerous luminal and abluminal protein transporters, solute and ion transporters and efflux transporters strictly controls the entry and exit of molecules and metabolic by-products across cells along the transcellular pathway. This results in low paracellular and transcellular permeability. While endothelial cells form the BBB, it is the complex interactions of astrocytes, pericytes, neurons, microglia, and the acellular basement membrane forming the neurovascular unit, that are essential for the development of barrier properties, neurovascular coupling, angiogenesis and neurogenesis. Tight junctions, located closest to the apical membrane, maintain BBB integrity by preventing the diffusion of proteins between luminal and abluminal membrane compartments and limit paracellular diffusion of ions and solutes across the BBB. The integral components of the tight junction are claudins-1, -3, -5 and -12 and occludin which are directly responsible for determining permeability of the tight junction. Therefore, the degree of tightness is determined by interactions of tight junction family members on endothelial cells.

Claudin-5 consists of 4 transmembrane domains (TM), a short NH₂ terminus, two extracellular loops (ECL), a short intracellular loop and a longer COOH terminus. ECL1 contains a disulphide bond and ion binding site as well as a highly conserved signature motif. The long COO-1 terminus contains the PDZ binding motif for interactions with scaffolding proteins including ZO-1, -2 and -3. Additionally, the COOH terminus contains trafficking and phosphorylation residues. Claudin-5 is localised specifically to the endothelial cell layer in the brain, lung, liver, kidney and skin while it is expressed in the epithelial cell layer in the stomach, with highest expression levels in the brain and lung. Claudin-5 expression is regulated by several upstream signaling pathways at the transcriptional and post-translational levels. Additionally, regulation of claudin-5 and of tight junction properties in general occurs via physical interactions with cytoplasmic scaffolding proteins and interactions with proteins on the same (cis) or adjacent cells (trans). Together, these processes determine claudin-5 assembly, remodeling and degradation.

The paracellular sealing function of claudin-5 stems from its association with claudin proteins on neighboring endothelial cells. Tight junctions provide one mechanism of cell to cell adhesion through their shared interactions with other tight junction strands on opposing cell membranes at so called “kissing points” to reduce the intercellular distance to almost zero. In essence, tight junctions form a mechanical link between individual endothelial cells to maintain the structural integrity of the vasculature and to prevent the diffusion of solutes and ions through the intercellular space. The result of this tight construction is a high electrical resistance of ˜1500-2000 Ω·cm².

At the blood-brain barrier, claudin-5 is the most enriched tight junction protein and its dysfunction has been implicated in neurodegenerative disorders such as Alzheimer's disease, neuroinflammatory disorders such as multiple sclerosis as well as psychiatric disorders including depression and schizophrenia (see, Greene, C., Hanley. N. & Campbell, M. Claudin-5: gatekeeper of neurological function. Fluids Barriers CNS 16, 3 (2019) doi.org/10.1186/s12987-019-0123-z; incorporated herein by reference). Dynamic tight junction remodeling has been reported in various diseases. Breakdown of the blood-brain barrier (BBB) and loss and mis-localization of tight junction proteins leads to immune cell entry to the central nervous system (CNS) in multiple sclerosis, this results in neuroinflammation, neurodegeneration and disease progression and transendothelial migration (TEM) of peripheral blood leukocytes. Claudin-5 positive extracellular vesicles (EV) can bind to blood leukocytes to potentially facilitate TEM of leukocytes into the CNS. BBB breakdown also leads to the perivascular accumulation of plasma-derived proteins such as fibrinogen, albumin and immunoglobulin G (IgG) that is found in humans with temporal lobe epilepsy as well as in rodents injected with the seizure-inducing agent kainic acid. In rodents, glutamate released from neurons and astrocytes can bind to N-Methyl-D-aspartate receptors (NMDAR) on the brain endothelium and regulate tight junction proteins claudin-5 and occludin via upregulation of matrix metalloproteinases (MMP). Extravasation of red blood cells (RBC) following traumatic brain injury releases toxic haemoglobin and free iron culminating in generation of reactive oxygen species (ROS). Extravasation of fibrinogen and albumin activates microglia leading to secretion of MMP and basement membrane (BM) degeneration. Dashed boxes display the signaling pathways and molecules that regulate expression of claudin-5 and subsequent disassembly of the tight junction protein complexes that facilitates paracellular BBB permeability of blood-derived molecules. Thus, by regulating levels of claudin-5, it is possible to abrogate disease symptoms in many of these disorders.

Disclosed herein for the first time is a pharmacologic agent (P7C3 class of compounds) that is shown to elevate Claudin-5 in vivo. Thus, elevating endothelial levels of claudin-5 by treatment with P7C3 molecules can be used in a wide range of neuropsychiatric conditions, including the following (with references that support a role of diminished Claudin-5 in the pathophysiology of the condition; all incorporated herein by reference):

Stroke
Koto T, et al. Hypoxia disrupts the barrier function of neural blood vessels through changes in the expression of claudin-5 in endothelial cells. Am J Pathol. 2007; 170(4):1389-97.
Yang Y, et al. Matrix metalloproteinase-mediated disruption of tight junction proteins in cerebral vessels is reversed by synthetic matrix metalloproteinase inhibitor in focal ischemia in rat. J Cereb Blood Flow Metab. 2007; 27(4):697-709.
Knowland D, et al. Stepwise recruitment of transcellular and paracellular pathways underlies blood-brain barrier breakdown in stroke. Neuron. 2014; 82(3):603-17

Traumatic Brain Injury
Baskaya M K, et al. The biphasic opening of the blood-brain barrier in the cortex and hippocampus after traumatic brain injury in rats. Neurosci Lett. 1997; 226(1):33-6.
Nag S, Venugopalan R, Stewart D J. Increased caveolin-1 expression precedes decreased expression of occludin and claudin-5 during blood-brain barrier breakdown. Acta Neuropathol. 2007; 114(5):459-69.
Campbell M, et al. Targeted suppression of claudin-5 decreases cerebral oedema and improves cognitive outcome following traumatic brain injury. Nat Commun. 2012; 3:849.
Doherty C P, et al. Blood-brain barrier dysfunction as a hallmark pathology in chronic traumatic encephalopathy. J Neuropathol Exp Neurol. 2016; 75(7):656-62.
Farrell M, et al. Blood-brain barrier dysfunction in a boxer with chronic traumatic encephalopathy and schizophrenia. Clin Neuropathol. 2018. doi.org/10.5414/NP301130.

Velocardial Facial Syndrome
Morita K, et al. Endothelial claudin: claudin-5/Tmvcf constitutes tight junction strands in endothelial cells. J Cell Biol. 1999; 147(1):185-94
Sun Z Y, et al. The CLDN5 locus may be involved in the vulnerability to schizophrenia. Eur Psychiatry. 2004; 19(6):354-7.
Wu N, et al. A weak association of the CLDN5 locus with schizophrenia in Chinese case-control samples. Psychiatry Res. 2010; 178(1):223.
Ye L, et al. Further study of a genetic association between the CLDN5 locus and schizophrenia. Schizophr Res. 2005; 75(1):139-41.

Epilepsy
Kim J Y, et al. ETB receptor-mediated MMP-9 activation induces vasogenic edema via ZO-1 protein degradation following status epilepticus. Neuroscience. 2015; 304:355-67.
Liu J Y, et al. Neuropathology of the blood-brain barrier and pharmacoresistance in human epilepsy. Brain. 2012; 135(Pt 10):3115-33.
Rigau V, et al. Angiogenesis is associated with blood-brain barrier permeability in temporal lobe epilepsy. Brain. 2007; 130(Pt 7):1942-56.
Rempe R G, et al. Matrix metalloproteinase-mediated blood-brain barrier dysfunction in epilepsy. J Neurosci. 2018; 38(18):4301-15.

Alzheimer's Disease (AD)
Comparative transcriptomics of choroid plexus in Alzheimer's disease, frontotemporal dementia and Huntington's disease: implications for CSF homeostasis. Stopa E G, Tanis K Q, Miller M C, Nikonova E V, Podtelezhnikov A A, Finney E M, Stone D J, Camargo L M, Parker L, Verma A, Baird A, Donahue J E, Torabi T, Eliceiri B P, Silverberg G D, Johanson C E. Fluids Barriers CNS. 2018 May 31; 15(1):18. doi: 10.1186/s12987-018-0102-9.PMID: 29848382
Selective loss of cortical endothelial tight junction proteins during Alzheimer's disease progression. Yamazaki Y, Shinohara M, Shinohara M, Yamazaki A, Murray M E, Liesinger A M, Heckman M G, Lesser E R, Parisi J E, Petersen R C, Dickson D W, Kanekiyo T, Bu G. Brain. 2019 Apr. 1; 142(4):1077-1092. doi: 10.1093/brain/awz011.PMID: 30770921

Glioblastoma

Multiple Sclerosis
Paul D, et al. Novel 3D analysis of Claudin-5 reveals significant endothelial heterogeneity among CNS microvessels. Microvasc Res. 2013; 86:1-10.
Lutz S E, et al. Caveolin 1 Is required for Th1 cell infiltration, but not tight junction remodeling, at the blood-brain barrier in autoimmune neuroinfammation. Cell Rep. 2017; 21(8):2104-17.
Grygorowicz T, Dabrowska-Bouta B, Struzynska L. Administration of an antagonist of P2X7 receptor to EAE rats prevents a decrease of expression of claudin-5 in cerebral capillaries. Purinergic Signal. 2018; 8:1-9.
Ni C, et al. Interferon-gamma safeguards blood-brain barrier during experimental autoimmune encephalomyelitis. Am J Pathol. 2014; 184(12):3308-20.
Lanz T V, et al. Protein kinase C3 as a therapeutic target stabilizing blood-brain barrier disruption in experimental autoimmune encephalomyelitis. Proc Natl Acad Sci USA. 2013; 110(36):14735-40.

Bipolar Disorder
Serum zonulin and claudin-5 levels in patients with bipolar disorder. Kιlιç F, Is̨ik Ü, Demirdas̨ A, Doğuç D K, Bozkurt M. J Affect Disord. 2020 Apr. 1; 266:37-42. doi: 10.1016/j.jad.2020.01.117. Epub 2020 Jan. 23.PMID: 32056901

Obsessive Compulsive Disorder
Serum zonulin and claudin-5 levels in children with obsessive-compulsive disorder. Işιk Ü, Aydoğan Avşar P, Aktepe E, Doğuç D K, Kιlιç F, Büyükbayram H Ï. Nord J Psychiatry. 2020 May-July; 74(5):346-351. doi: 10.1080/08039488.2020.1715474. Epub 2020 Jan. 21.PMID: 31961248

ADHD
Serum zonulin and claudin-5 levels in children with attention-deficit/hyperactivity disorder. Aydoğan Avşar P, Işιk Ü, Aktepe E, Kιlιç F, Doğuç D K, Büyükbayram H İ. Int J Psychiatry Clin Pract. 2020 Aug. 6:1-7. doi: 10.1080/13651501.2020.1801754. Online ahead of print. PMID: 32757874

Depression
Schachtrup C, et al. Fibrinogen inhibits neurite outgrowth via beta 3 integrin-mediated phosphorylation of the EGF receptor. Proc Natl Acad Sci USA. 2007; 104(28):11814-9.
Santha P, et al. Restraint stress-induced morphological changes at the blood-brain barrier in adult rats. Front Mol Neurosci. 2015; 8:88.
Lee S, et al. Real-time in vivo two-photon imaging study reveals decreased cerebro-vascular volume and increased blood-brain barrier permeability in chronically stressed mice. Sci Rep. 2018; 8(1):13064.
Northrop N A, Yamamoto B K. Persistent neuroinfammatory efects of serial exposure to stress and methamphetamine on the blood-brain barrier. J Neuroimmune Pharmacol. 2012; 7(4):951-68.
Social stress induces neurovascular pathology promoting depression. Menard C, Pfau M L, Hodes G E, Kana V, Wang V X, Bouchard S, Takahashi A, Flanigan M E, Aleyasin H, LeClair K B, Janssen W G, Labonté B, Parise E M, Lorsch Z S, Golden S A, Heshmati M, Tamminga C, Turecki G, Campbell M, Fayad Z A, Tang C Y, Merad M, Russo S J. Nat Neurosci. 2017 December; 20(12):1752-1760. doi: 10.1038/s41593-017-0010-3. Epub 2017 Nov. 13.PMID: 29184215
Molecular adaptations of the blood-brain barrier promote stress resilience vs. depression. Dudek K A, Dion-Albert L, Lebel M, LeClair K, Labrecque S, Tuck E, Ferrer Perez C, Golden S A, Tamminga C, Turecki G, Mechawar N, Russo S J, Menard C. Proc Natl Acad Sci USA. 2020 Feb. 11; 117(6):3326-3336. doi: 10.1073/pnas.1914655117. Epub 2020 Jan. 23.PMID: 31974313

Pain Disorders
Huber J D, et al. Infammatory pain alters blood-brain barrier permeability and tight junctional protein expression. Am J Physiol Heart Circ Physiol. 2001; 280(3):H1241-8
Chronic infammatory pain leads to increased blood-brain barrier permeability and tight junction protein alterations. Am J Physiol Heart Circ Physiol. 2005; 289(2):H738-43.
Brooks T A, et al. Biphasic cytoarchitecture and functional changes in the BBB induced by chronic infammatory pain. Brain Res. 2006; 1120(1):172-82.
Campos C R, et al. Nociceptive inhibition prevents infammatory pain induced changes in the blood-brain barrier. Brain Res. 2008; 1221:6-13.
Yucel M, et al. Serum levels of endocan, claudin-5 and cytokines in migraine. Eur Rev Med Pharmacol Sci. 2016; 20(5):930-6.
Cottier K E, et al. Loss of blood-brain barrier integrity in a KCl-induced model of episodic headache enhances CNS drug delivery. eNeuro. 2018. doi.org/10.1523/ENEURO.0116-18.2018

Schizophrenia
Farrell M, et al. Blood-brain barrier dysfunction in a boxer with chronic traumatic encephalopathy and schizophrenia. Clin Neuropathol. 2018. doi.org/10.5414/NP301130.
Nishiura K, et al. PKA activation and endothelial claudin-5 breakdown in the schizophrenic prefrontal cortex. Oncotarget. 2017; 8(55):93382-91.
Dose-dependent expression of claudin-5 is a modifying factor in schizophrenia. Greene C, Kealy J, Humphries M M, Gong Y, Hou J, Hudson N, Cassidy L M, Martiniano R, Shashi V, Hooper S R, Grant G A, Kenna P F, Norris K, Callaghan C K, Islam M D, O'Mara S M, Najda Z, Campbell S G, Pachter J S, Thomas J, Williams N M, Humphries P, Murphy K C, Campbell M. Mol Psychiatry. 2018 November; 23(11):2156-2166. doi: 10.1038/mp.2017.156. Epub 2017 Oct. 10.PMID: 28993710
Serum zonulin and claudin-5 levels in patients with schizophrenia. Usta A, Kιlιç F, Demirdaş A, Işκk Ü, Doğuç D K, Bozkurt M. Eur Arch Psychiatry Clin Neurosci. 2020 Jun. 20. doi: 10.1007/s00406-020-01152-9. Online ahead of print. PMID: 32564127
PKA activation and endothelial claudin-5 breakdown in the schizophrenic prefrontal cortex. Nishiura K, Ichikawa-Tomikawa N, Sugimoto K, Kunii Y, Kashiwagi K, Tanaka M, Yokoyama Y, Hino M, Sugino T, Yabe H, Takahashi H, Kakita A, Imura T, Chiba H. Oncotarget. 2017 Oct. 16; 8(55):93382-93391. doi: 10.18632/oncotarget.21850. eCollection 2017 Nov. 7.PMID: 29212157
Breakdown of the Paracellular Tight and Adherens Junctions in the Gut and Blood Brain Barrier and Damage to the Vascular Barrier in Patients with Deficit Schizophrenia. Maes M, Sirivichayakul S, Kanchanatawan B, Vodjani A. Neurotox Res. 2019 August; 36(2):306-322. doi: 10.1007/s12640-019-00054-6. Epub 2019 May 10.PMID: 31077000
The CLDN5 locus may be involved in the vulnerability to schizophrenia. Sun Z Y, Wei J, Xie L, Shen Y, Liu S Z, Ju G Z, Shi J P, Yu Y Q, Zhang X, Xu Q, Hemmings G P. Eur Psychiatry. 2004 September; 19(6):354-7. doi: 10.1016/j.eurpsy.2004.06.007.PMID: 15363474
Association of a functional Claudin-5 variant with schizophrenia in female patients with the 22q11.2 deletion syndrome.
Guo Y, Singh L N, Zhu Y, Gur R E, Resnick A, Anderson S A, Alvarez J I. Schizophr Res. 2020 January; 215:451-452. doi: 10.1016/j.schres.2019.09.014. Epub 2019 Oct. 23

Heart Failure
Myocardial Contractile Dysfunction Is Present without Histopathology in a Mouse Model of Limb-Girdle Muscular Dystrophy-2F and Is Prevented after Claudin-5 Virotherapy. Milani-Nejad N, Schultz E J, Slabaugh J L, Janssen P M, Rafael-Fortney J A. Front Physiol. 2016 Dec. 6; 7:539. doi: 10.3389/fphys.2016.00539. eCollection 2016.PMID: 27999547

### Pharmaceutical Compositions

The term “pharmaceutically acceptable carrier” refers to a carrier or adjuvant that may be administered to a subject (e.g., a patient), together with a compound of the present disclosure, and which does not destroy the pharmacological activity thereof and is nontoxic when administered in doses sufficient to deliver a therapeutic amount of the compound.

Pharmaceutically acceptable carriers, adjuvants and vehicles that may be used in the compositions of the present disclosure include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts, or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds described herein.

The compositions for administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, losenges or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

The amount administered depends on the compound formulation, route of administration, etc. and is generally empirically determined in routine trials, and variations will necessarily occur depending on the target, the host, and the route of administration, etc. Generally, the quantity of active compound in a unit dose of preparation may be varied or adjusted from about 1, 3, 10 or 30 to about 30, 100, 300 or 1000 mg, according to the particular application. In a particular embodiment, unit dosage forms are packaged in a multipack adapted for sequential use, such as blisterpack, comprising sheets of at least 6, 9 or 12 unit dosage forms. The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

The following are examples (Formulations 1-4) of capsule formulations.

### Capsule Formulations

| | Formulation | Formulation | Formulation | Formulation |
|---|---|---|---|---|
| Capsule | 1 | 2 | 3 | 4 |
| Formulation | mg/capsule | mg/capsule | mg/capsule | mg/capsule |
| Compound | 100 | 400 | 400 | 200 |
| (solid | | | | |
| solution) | | | | |
| Silicon | 0.625 | 2.5 | 3.75 | 1.875 |
| Dioxide | | | | |
| Magnesium | 0.125 | 0.5 | 0.125 | 0.625 |
| Stearate NF2 | | | | |
| Croscarmellose | 11.000 | 44.0 | 40.0 | 20.0 |
| Sodium NF | | | | |
| Pluronic | 6.250 | 25.0 | 50.0 | 25.0 |
| F68 NF | | | | |
| Silicon | 0.625 | 2.5 | 3.75 | 1.875 |
| Dioxide NF | | | | |
| Magnesium | 0.125 | 0.5 | 1.25 | 0.625 |
| Stearate NF | | | | |
| Total | 118.750 | 475.00 | 475.00 | 475.00 |
| Capsule Size | No. 4 | No. 0 | No. 0 | No. 2 |

### Preparation of Solid Solution

Crystalline compound (80 g/batch) and the povidone (NF K29/32 at 160 g/batch) are dissolved in methylene chloride (5000 mL). The solution is dried using a suitable solvent spray dryer and the residue reduced to fine particles by grinding. The powder is then passed through a 30 mesh screen and confirmed to be amorphous by x-ray analysis.

The solid solution, silicon dioxide and magnesium stearate are mixed in a suitable mixer for 10 minutes. The mixture is compacted using a suitable roller compactor and milled using a suitable mill fitted with 30 mesh screen. Croscarmellose sodium, Pluronic F68 and silicon dioxide are added to the milled mixture and mixed further for 10 minutes. A premix is made with magnesium stearate and equal portions of the mixture. The premix is added to the remainder of the mixture, mixed for 5 minutes and the mixture encapsulated in hard shell gelatin capsule shells.

### Use

In one aspect, methods for treating (e.g., controlling, relieving, ameliorating, alleviating, or slowing the progression of) or methods for preventing (e.g., delaying the onset of or reducing the risk of developing) one or more diseases, disorders, or conditions. The methods include administering to the subject an effective amount of any compound described herein or a salt (e.g., a pharmaceutically acceptable salt) thereof as defined anywhere herein to the subject.

In another aspect, the use of any compound described herein or a salt (e.g., a pharmaceutically acceptable salt) thereof as defined anywhere herein in the preparation of, or for use as, a medicament for the treatment (e.g., controlling, relieving, ameliorating, alleviating, or slowing the progression of) or prevention (e.g., delaying the onset of or reducing the risk of developing) of one or more diseases, disorders, or conditions.

In embodiments, the one or more diseases, disorders, or conditions can include neuropathies, nerve trauma, and neurodegenerative diseases. In embodiments, the one or more diseases, disorders, or conditions can be diseases, disorders, or conditions caused by, or associated with aberrant (e.g., insufficient) neurogenesis (e.g., aberrant hippocampal neurogenesis as is believed to occur in neuropsychiatric diseases) or accelerated death of existing neurons. Examples of the diseases, disorders, or conditions include, but are not limited to, DNA-damaging agent (e.g., anthracycline) mediated cardiotoxicity, schizophrenia, major depression, bipolar disorder, normal aging, epilepsy, traumatic brain injury and/or a visual symptom associated therewith, post-traumatic stress disorder, Parkinson's disease, Alzheimer's disease, Down syndrome, spinocerebellar ataxia, amyotrophic lateral sclerosis, Huntington's disease, stroke, radiation therapy, chronic stress, abuse of a neuro-active drug, retinal degeneration, spinal cord injury, peripheral nerve injury, physiological weight loss associated with various conditions, cognitive decline and/or general frailty associated with normal aging and/or chemotherapy, chemotherapy induced neuropathy, concussive injury, crush injury, peripheral neuropathy, diabetic neuropathy, post-traumatic headache, multiple sclerosis, retinal degeneration and dystrophy (such as Leber congenital amaurosis, retinitis pigmentosa, cone-rod dystrophy, microphthalmia, anophthalmia, myopia, and hyperopia), spinal cord injury, traumatic spinal cord injury, peripheral nerve injury (such as peripheral nerve crush injury, neonatal brachial plexus palsy, and traumatic facial nerve palsy), retinal neuronal death related diseases (such as glaucoma and age related macular degeneration, diabetic retinopathy, retinal blood vessel occlusions, retinal medication toxicity (such as what amino glycosides or plaquenil can cause), retinal trauma (e.g., post-surgical), retinal infections, choroidal dystrophies, retinal pigmentary retinopathies, inflammatory and cancer mediated auto immune diseases that result in retinal neuronal cell death), Autism, Stargardt disease, Kearns-Sayre syndrome, Pure neurosensory deafness, Hereditary hearing loss with retinal diseases, Hereditary hearing loss with system atrophies of the nervous system, Progressive spinal muscular atrophy, Progressive bulbar palsy, Primary lateral sclerosis, Hereditary forms of progressive muscular atrophy and spastic paraplegia, Frontotemporal dementia, Dementia with Lewy bodies, Corticobasal degeneration, Progressive supranuclear palsy, Prion disorders causing neurodegeneration, Multiple system atrophy (olivopontocerebellar atrophy), Hereditary spastic paraparesis, Friedreich ataxia, Non-Friedreich ataxia, Spinocerebellar atrophies, Amyloidoses, Metabolic-related (e.g., Diabetes) neurodegenerative disorders, Toxin-related neurodegenerative disorders, Multiple sclerosis, Charcot Marie Tooth, Diabetic neuropathy, Metabolic neuropathies, Endocrine neuropathies, Orthostatic hypotension, Creutzfeldt-Jacob Disease, Primary progressive aphasia, Frontotemporal Lobar Degeneration, Cortical blindness, Shy-Drager Syndrome (Multiple, System Atrophy with Orthostatic Hypotension), Diffuse cerebral cortical atrophy of non-Alzheimer type, Lewy-body dementia, Pick disease (lobar atrophy), Thalamic degeneration, Mesolimbocortical dementia of non-Alzheimer type, Nonhuntingtonian types of chorea and dementia, Cortical-striatal-spinal degeneration, Dementia-Parkinson-amyotrophic lateral sclerosis complex, Cerebrocerebellar degeneration, Cortico-basal ganglionic degeneration, Familial dementia with spastic paraparesis or myoclonus, and Tourette syndrome.

The resultant promotion of neurogenesis or survival of existing neurons (i.e., a resultant promotion of survival, growth, development, function and/or generation of neurons) may be detected directly, indirectly or inferentially from an improvement in, or an amelioration of one or more symptoms of the disease or disorder caused by or associated with aberrant neurogenesis or survival of existing neurons. Suitable assays which directly or indirectly detect neural survival, growth, development, function and/or generation are known in the art, including axon regeneration in rat models (e.g. Park et al., Science. 2008 Nov. 7; 322:963-6), nerve regeneration in a rabbit facial nerve injury models (e.g. Zhang et al., J Transl Med. 2008 Nov. 5; 6(1):67); sciatic nerve regeneration in rat models (e.g. Sun et al., Cell Mol Neurobiol. 2008 Nov. 6); protection against motor neuron degeneration in mice (e.g. Poesen et al., J. Neurosci. 2008 Oct. 15; 28(42):10451-9); rat model of Alzheimer's disease, (e.g. Xuan et al., Neurosci Lett. 2008 Aug. 8; 440(3):331-5); animal models of depression (e.g. Schmidt et al., Behav Pharmacol. 2007 September; 18(5-6):391-418; Krishnan et al., Nature 2008, 455, 894-902); and/or those exemplified herein.

### Administration

The compounds and compositions described herein can, for example, be administered orally, parenterally (e.g., subcutaneously, intracutaneously, intravenously, intramuscularly, intraarticularly, intraarterially, intrasynovially, intrasternally, intrathecally, intralesionally and by intracranial injection or infusion techniques), by inhalation spray, topically, rectally, nasally, buccally, vaginally, via an implanted reservoir, by injection, subdermally, intraperitoneally, transmucosally, or in an ophthalmic preparation, with a dosage ranging from about 0.01 mg/kg to about 1000 mg/kg, (e.g., from about 0.01 to about 100 mg/kg, from about 0.1 to about 100 mg/kg, from about 1 to about 100 mg/kg, from about 1 to about 10 mg/kg) every 4 to 120 hours, or according to the requirements of the particular drug. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, N.Y., 537 (1970). In certain embodiments, the compositions are administered by oral administration or administration by injection. The methods herein contemplate administration of an effective amount of compound or compound composition to achieve the desired or stated effect. Typically, the pharmaceutical compositions of the present disclosure will be administered from about 1 to about 6 times per day or alternatively, as a continuous infusion. Such administration can be used as a chronic or acute therapy.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of the present disclosure may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

In some embodiments, the compounds described herein can be co-administered with one or more other therapeutic agents. In certain embodiments, the additional agents may be administered separately, as part of a multiple dose regimen, from the compounds of the present disclosure (e.g., sequentially, e.g., on different overlapping schedules with the administration of one or more compounds disclosed herein (including any subgenera or specific compounds thereof)). In other embodiments, these agents may be part of a single dosage form, mixed together with the compounds of the present disclosure in a single composition. In still another embodiment, these agents can be given as a separate dose that is administered at about the same time that one or more compounds disclosed herein (including any subgenera or specific compounds thereof) are administered (e.g., simultaneously with the administration of one or more compounds disclosed herein (including any subgenera or specific compounds thereof)). When the compositions of the present disclosure include a combination of a compound described herein and one or more additional therapeutic or prophylactic agents, both the compound and the additional agent can be present at dosage levels of between about 1 to 100%, and more preferably between about 5 to 95% of the dosage normally administered in a monotherapy regimen.

The compositions of the present disclosure may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions of the present disclosure may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The compositions of the present disclosure may also be administered in the form of suppositories for rectal administration. These compositions can be prepared by mixing a compound of the present disclosure with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the compositions of the present disclosure is useful when the desired treatment involves areas or organs readily accessible by topical application. For application topically to the skin, the composition should be formulated with a suitable ointment containing the active components suspended or dissolved in a carrier. Carriers for topical administration of the compounds of the present disclosure include, but are not limited to, mineral oil, liquid petroleum, white petroleum, propylene glycol, polyoxyethylene polyoxypropylene compound, emulsifying wax and water. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The compositions of the present disclosure may also be topically applied to the lower intestinal tract by rectal suppository formulation or in a suitable enema formulation.

In some embodiments, topical administration of the compounds and compositions described herein may be presented in the form of an aerosol, a semi-solid pharmaceutical composition, a powder, or a solution. By the term “a semi-solid composition” is meant an ointment, cream, salve, jelly, or other pharmaceutical composition of substantially similar consistency suitable for application to the skin. Examples of semi-solid compositions are given in Chapter 17 of The Theory and Practice of Industrial Pharmacy, Lachman, Lieberman and Kanig, published by Lea and Febiger (1970) and in Remington's Pharmaceutical Sciences, 21st Edition (2005) published by Mack Publishing Company, which is incorporated herein by reference in its entirety.

Topically-transdermal patches are also included in the present disclosure. Also within the present disclosure is a patch to deliver active chemotherapeutic combinations herein. A patch includes a material layer (e.g., polymeric, cloth, gauze, bandage) and the compound delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

The compositions of the present disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compounds disclosed herein and an additional agent (e.g., a therapeutic agent) can be administered using any of the routes of administration described herein. In some embodiments, a composition having the compound disclosed herein and an additional agent (e.g., a therapeutic agent) can be administered using an implantable device. Implantable devices and related technology are known in the art and are useful as delivery systems where a continuous or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in the present disclosure. For example, timed-release formulations based on polymer technologies, sustained-release techniques and encapsulation techniques (e.g., polymeric, liposomal) can also be used for delivery of the compounds and compositions delineated herein.

The present disclosure will be further described in the following examples. It should be understood that these examples are for illustrative purposes only and are not to be construed as limiting the present disclosure in any manner.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); wt, wild type; and the like.

### Example 1: P7C3-A20 Protects the Blood-Brain Barrier after Systemic Inflammation or Traumatic Brain Injury (TBI), and Treatment One Year after TBI Arrests Chronic Neurodegeneration and Restores Cognition

### Summary

Chronic neurodegeneration in survivors of traumatic brain injury (TBI) is a major cause of morbidity, with no effective therapies to mitigate this progressive and debilitating form of nerve cell death. Here, we report that pharmacologic restoration of the blood-brain barrier (BBB), 12 months after murine TBI, is associated with arrested axonal neurodegeneration and cognitive recovery; benefits that persisted for months after treatment cessation. Recovery was achieved by 30 days of once daily administration of P7C3-A20, a compound that stabilizes cellular energy levels. Four months after P7C3-A20, electron microscopy revealed full repair of TBI-induced breaks in cortical and hippocampal BBB endothelium. Immunohistochemical staining identified additional benefits of P7C3-A20, including restoration of normal BBB endothelium length, increased brain capillary pericyte density, increased expression of BBB tight junction proteins, reduced brain infiltration of immunoglobulin, and attenuated neuroinflammation. These changes were accompanied by cessation of TBI-induced chronic axonal degeneration. Specificity for P7C3-A20 action on the endothelium was confirmed by protection of cultured human brain microvascular endothelial cells from hydrogen peroxide-induced cell death, as well as preservation of BBB integrity in mice after exposure to toxic levels of lipopolysaccharide. P7C3-A20 also protected mice from BBB degradation after acute TBI. Collectively, our results provide novel insights into the pathophysiologic mechanisms behind chronic neurodegeneration after TBI, along with a putative treatment strategy. Because TBI increases the risks of other forms of neurodegeneration involving BBB deterioration, (e.g. Alzheimer's disease, Parkinson's disease, vascular dementia, chronic traumatic encephalopathy), P7C3-A20 may have wide-spread clinical utility in the setting of neurodegenerative conditions.

### Results

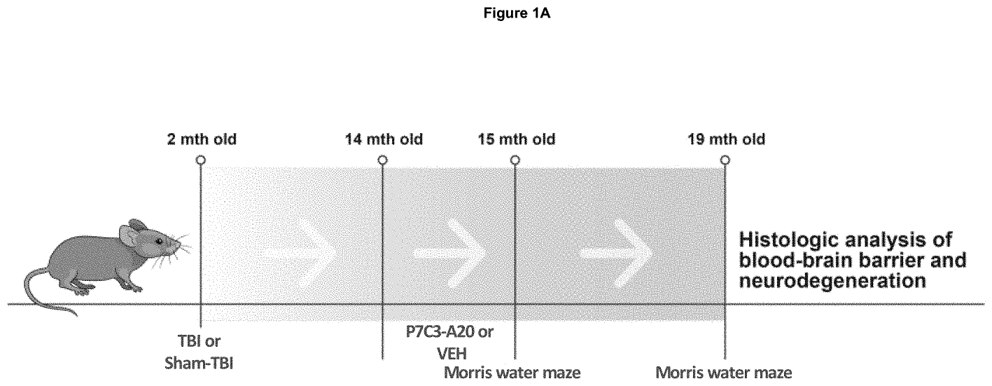

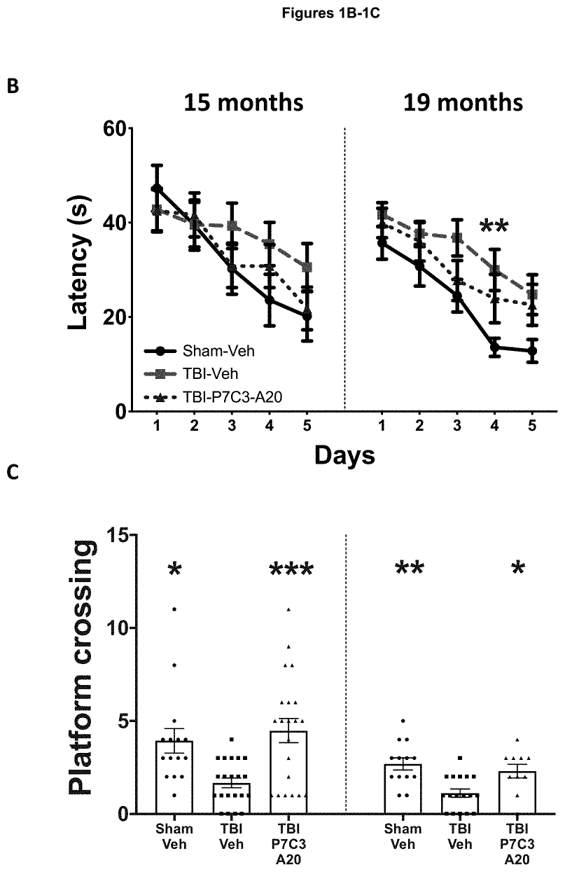

Recent work elegantly summarized by Sandsark et al (17) has identified injury to the neurovascular unit (NVU) as a potential driving force for chronic neurodegeneration after TBI. Damage to the endothelial lining of the brain microvessels is observed in both human patients and mouse TBI models (18), resulting in persistent deterioration of the blood-brain barrier (BBB) and chronic brain inflammation (19,20). Here, we have studied this chronic condition in a murine model of multimodal TBI (mmTBI) that entails jet-flow exposure in an overpressure chamber to produce globally compressive forces along with a variable amount of acceleration-deceleration and early blast wave exposure. This laboratory model produces neurodegeneration and neurobehavioral deficits reminiscent of TBI in people (21-24). We treated and analyzed mice beginning one year after a single injury, as outlined in FIG. 1A. This late time point was selected because it represents approximately the midpoint of a typical mouse's lifespan, and thus can be considered a model of delaying initiation of treatment in people until decades after their TBI. This is important because of the great many people living today who are suffering from the chronic deficits of TBI.

Two-month old mice were subjected to TBI or sham-TBI and housed under standard conditions for one year. Mice were then administered either vehicle (TBI-Veh, Sham-Veh) or P7C3-A20 (10 mg×kg⁻¹×day⁻¹ IP; TBI-P7C3-A20) for four weeks. P7C3-A20 is an aminopropyl carbazole that elevates cellular nicotinamide adenine dinucleotide, enhances survival of adult-born young hippocampal neurons, and has been shown to preserve mature neurons and improve cognition in various models of nervous system disease and injury (25-45). Immediately after treatment completion, cognitive function in the 15-month old animals was evaluated through Morris water maze (MWM) testing. Initial task learning was equivalent between groups (FIG. 1B). However, significant memory deficits were recorded in the TBI-Veh group, while memory performance in the TBI-P7C3-A20 mice was equivalent to the control animals (Sham-Veh; FIG. 1C).

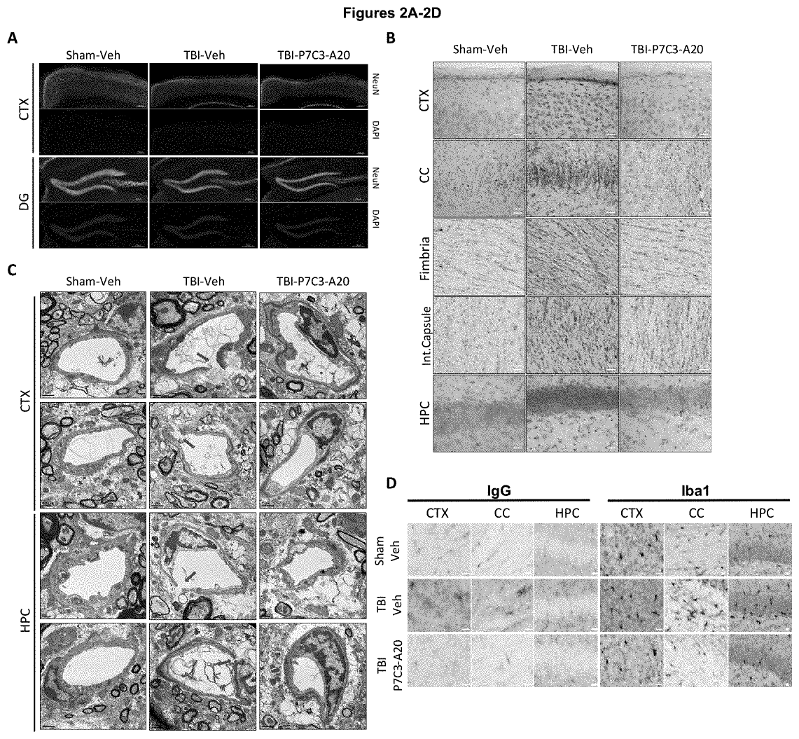

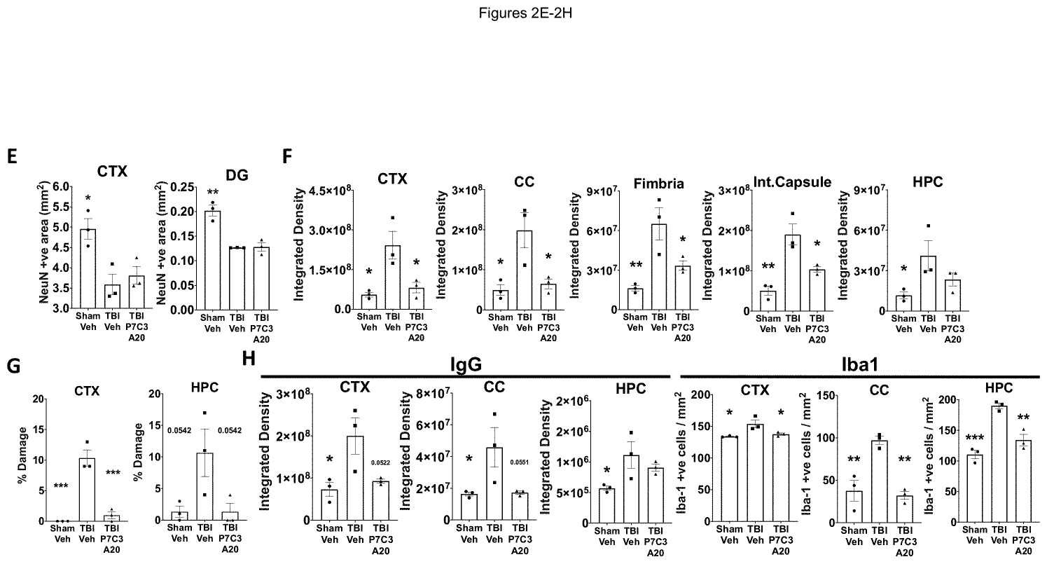

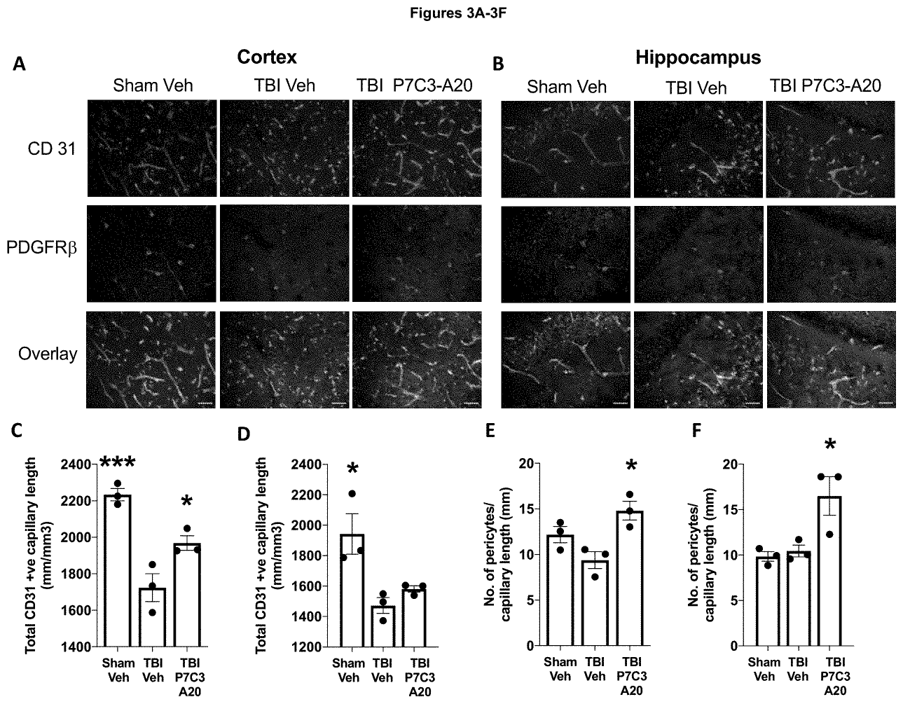

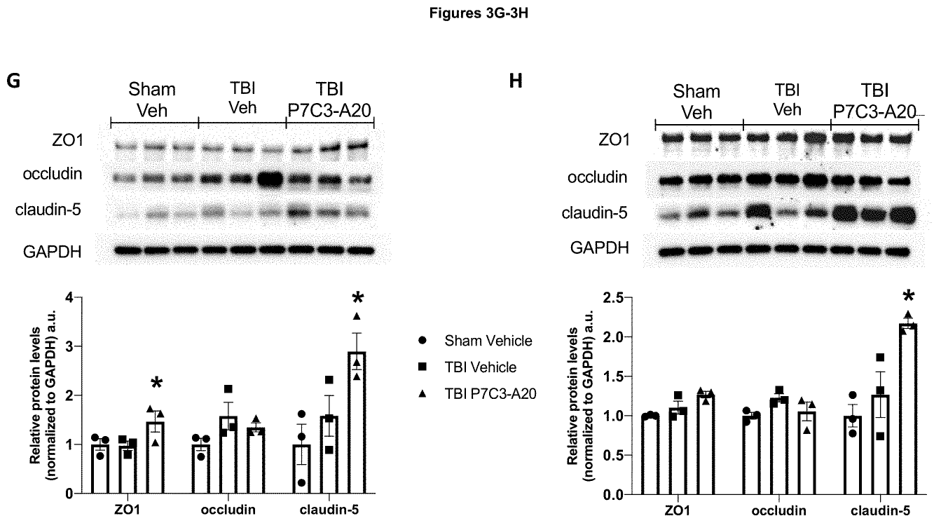

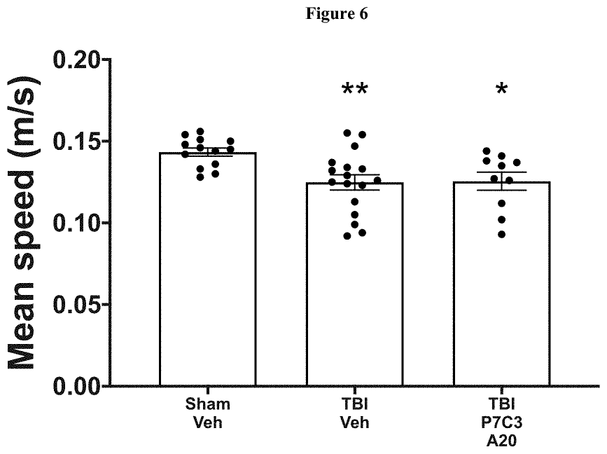

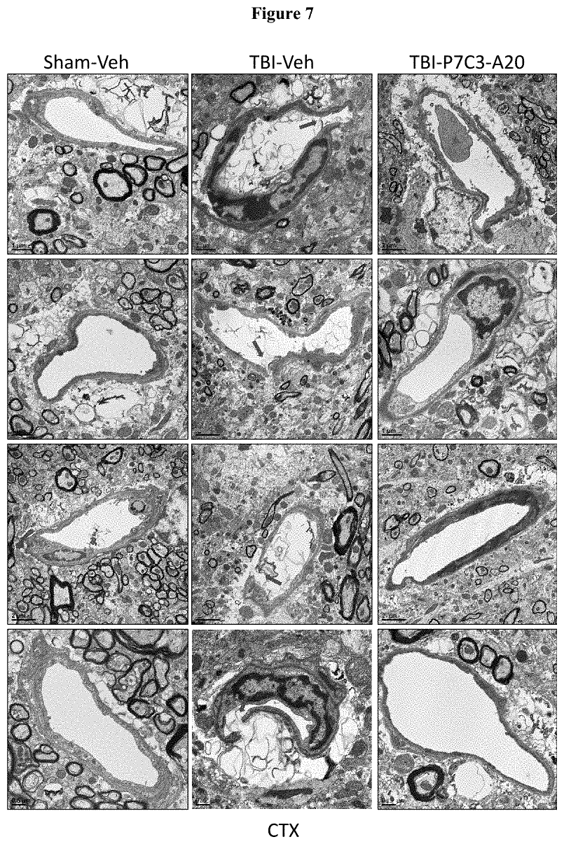

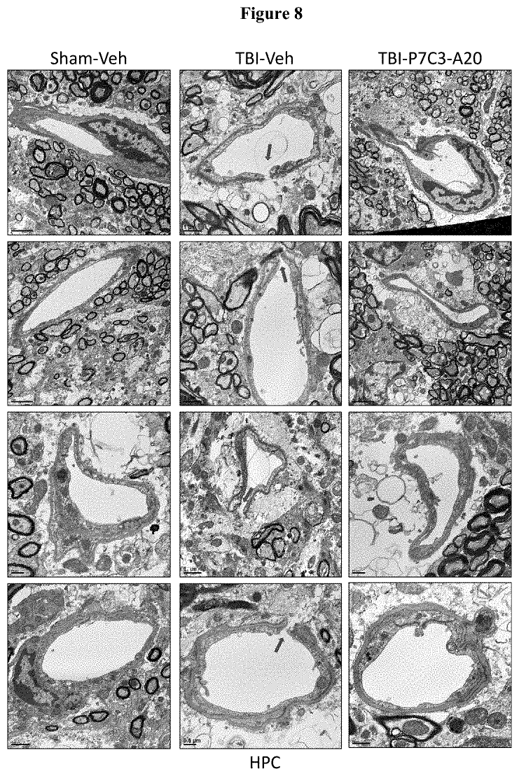

Animals were then housed under standard conditions with no treatment for 4 additional months (FIG. 1A). At 19 months of age (17 months post-injury), TBI-Veh mice exhibited impaired learning (FIG. 1B) and memory (FIG. 1C), whereas TBI-P7C3-A20 mice again performed as well as Sham-Veh animals (FIGS. 1B,1C). TBI-dependent motor slowing was not corrected (FIG. 6). While similar degrees of hippocampal and cortical neuronal cell loss were observed in both TBI groups (FIGS. 2A,2E), chronically-active neurodegeneration (as quantified by silver staining of axonal degeneration) was arrested by P7C3-A20 treatment (FIGS. 2B,2F). Assessment of the NVU by transmission electron microscopy revealed cortical and hippocampal BBB capillary endothelium breaks in TBI-Veh mice that were absent in Sham-Veh and TBI-P7C3-A20 treated animals (FIGS. 2C,2G, FIGS. 7,8). Immunohistochemical examination conducted in parallel showed that the prior P7C3-A20 treatment had also reduced infiltration of peripheral IgG (a marker of BBB degradation) and decreased the number of Iba1+cells (a marker of activated microglia in neuroinflammation; FIGS. 2D,2H). P7C3-A20 also restored BBB endothelium length toward that seen in Sham-Veh brains (FIGS. 3A-3D). Further benefits of P7C3-A20 on the BBB included increases in capillary pericyte density (FIGS. 3A,3B,3E,3F) and increases in the expression of BBB tight junction proteins claudin-5 in the cortex and hippocampus, and of zona occludens-1 in the cortex (FIGS. 3G,3H).

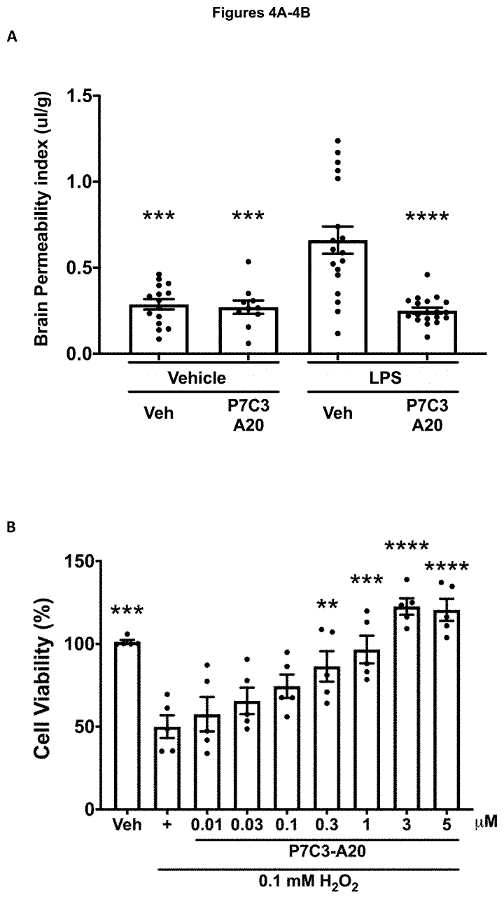

To confirm that P7C3-A20 was directly protecting brain endothelial cells, we dosed separate cohorts of mice with lipopolysaccharide (LPS), which damages the BBB (46). Twelve hours after inoculation, the treatment group received a single dose of P7C3-A20 (10 mg/kg IP). BBB integrity was assessed in both groups 12 h later by quantifying CNS permeation of fluorescent-conjugated 3 kD dextran (47). Notably, LPS-mediated entry of dextran was blocked in the P7C3-A20-treated mice (FIG. 4A). P7C3-A20 also directly protected cultured human microvascular endothelial cells from hydrogen-peroxide-mediated cell death (FIG. 4B), providing further evidence of this aminopropyl carbazole's ability to preserve endothelial health and function.

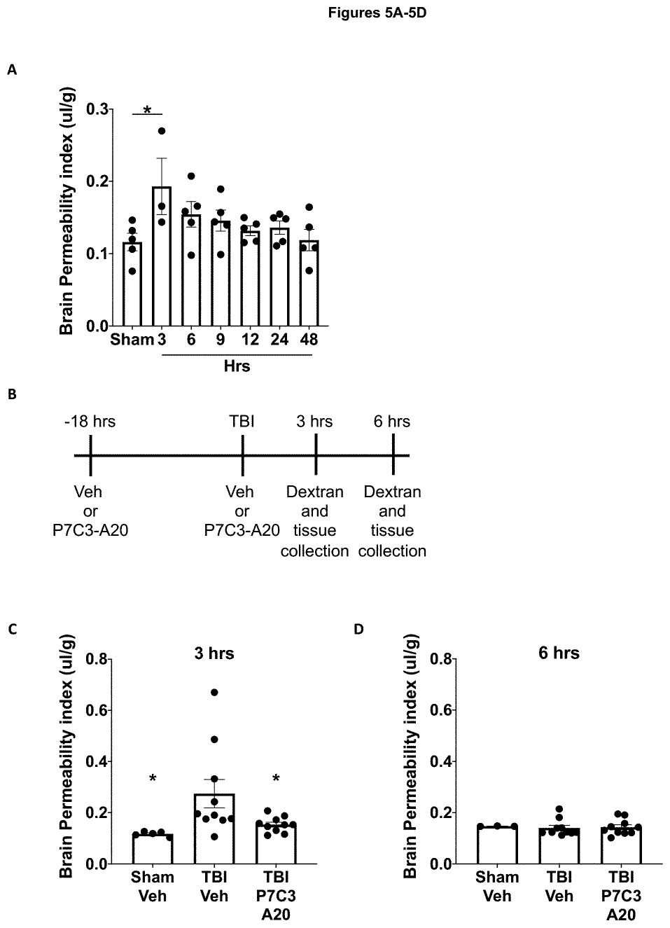

Lastly, we wondered whether treatment with P7C3-A20 would protect the BBB in acute TBI as well. Both we and others (39,48-53) have previously observed in different models of TBI that acute disruption of the BBB returns to normal at variable times after injury. Thus, we first established the pattern of acute BBB degradation in our model of mmTBI. As shown in FIG. 5A, CNS permeation of fluorescent-conjugated 3 kD dextran approximately doubled 3 hours after TBI, and returned to sham levels at subsequent time points of 6, 9, 12, 24, and 48 hours. We then treated mice with either Vehicle or P7C3-A20 (10 mg/kg IP) 18 hours before TBI and again at the time of TBI (FIG. 5B). Here, TBI-Veh mice showed approximately doubled BBB permeability compared to Sham-Veh mice at the 3 hour time point, as expected from the previous time course experiment, and TBI-P7C3-A20 mice were fully protected from this BBB degradation (FIG. 5C). At the 6 hour time point, none of the groups showed any impairment in BBB function (FIG. 5D), as expected.

### Discussion

Proper CNS function is critically dependent on an extensive vascular network that works in concert with multiple cell types to form the NVU. The NVU rapidly responds to the changing metabolic needs of the brain while also protecting the brain parenchyma from exposure to injurious agents via the BBB. The BBB prevents entry of peripheral toxins while also mediating the removal of proteins and other substances from the brain parenchyma. Through continuous crosstalk, the NVU forms an integrated system of neurovascular coupling that ensures optimal supply of oxygen and micronutrients across the BBB consonant with the metabolic demand that varies with neuronal activity. Disruption of the NVU and the BBB that it maintains can harm brain health, potentially leading to neurodegeneration (54,55). Here, we show that deterioration of brain microvascular endothelial cells accompanies chronic axonal degeneration one year after TBI in mice. This is consistent with human studies of chronic TBI. For example, in one study, 47% of brains from long-term TBI survivors (up to 47 years after injury) showed histologic evidence of BBB deterioration (56). Furthermore, decreased expression of claudin-1 and zona occludens-1 tight junction proteins and immunoglobulin extravasation into brain parenchyma was observed in brain tissue from a patient with chronic traumatic encephalopathy (57), and multiple imaging modalities have demonstrated chronically compromised cerebral blood flow dynamics in patients with chronic TBI (17).

We also show here for the first time that P7C3-A20 directly protects brain microvascular endothelial cells in vivo in mice and in cultured human cells. Taken together, our results suggest that BBB deterioration may be a major contributor to chronic neurodegeneration after remote TBI, and that its repair may halt this pathology. Although the mechanism and timing of damage to the BBB in our model remains to be further studied, our results provide a novel and rational foundation for developing treatments for patients suffering from chronic progressive neurodegeneration and cognitive dysfunction after remote TBI. This is critical for the field, as despite the enormous societal burden of chronic neurodegeneration after TBI, there are currently no medicines that prevent or slow this disease process. We note that it is currently not possible discern the potentially complementary beneficial effects of P7C3-A20 on both endothelial cells and neurons. Future pharmacologic tools that act selectively on endothelial cells or neurons might be able to resolve this question. At present, our results support the broad potential utility of a pharmacologic agent able to directly repair the BBB, a property that we demonstrate here is possessed by P7C3-A20.

Lastly, it is important to place into context how these results relate to the association between TBI and later neurodegenerative disease. Patients with AD and AD-related dementias frequently display increased BBB permeability (58), and TBI is a well-recognized major risk factor for AD and other dementias, including Parkinson's disease, vascular dementia, and chronic traumatic encepaholopathy (10,59-64). BBB deterioration has also been observed in these same neurodegenerative conditions (10,65,66). Recently, a blood biomarker of BBB deterioration was also proposed for AD (67), and the two-hit vascular hypothesis of AD proposes the BBB disruption is an initiating event in amyloid beta deposition in the brain (62). In support of this, human studies have identified alteration in BBB permeability at the earliest stages of cognitive decline and AD (68,69). How the trajectory of BBB dysfunction after TBI proceeds from acute impairment and recovery, to later chronic deterioration, is currently unknown and is an important question for the field. The astrocyte-derived lipid transport molecule apolipoprotein-e (apoE) is a major factor that regulates BBB permeability, via regulation of low density lipoprotein receptor-related protein 1-cyclophilin A-matrix metalloproteinase-9 signaling in pericytes at the NVU (70). It is interesting to note that the apoE4 allele, one of the strongest known genetic risk factors for AD (71), also impairs spontaneous BBB repair after TBI (48) and has been demonstrated as epidemiologically synergistic with TBI for increased risk of AD (72). In conclusion, we speculate that restoration of BBB integrity with P7C3-A20 after TBI could yield not only cognitive benefits but also reduce the otherwise increased risk for patients to develop other forms of neurodegenerative disease later in life.

### Materials and Methods

### Animals

Seven-week-old, male, C57BL/6J (Stock No: 000664) mice were obtained from Jackson Laboratories. All mice were maintained group-housed with water and food provided ad libitum under humidity, light (12 hours light/dark cycle), and temperature-controlled conditions. All animal procedures were performed in accordance with the protocol approved by the University of Iowa Institutional Animal Care and Use Committee and the Louis Stokes Cleveland VA Medical Center Institutional Animal Care and Use Committee.

### Drug Administration

P7C3-A20 was dissolved in 2.5% vol of DMSO, followed by addition of 10% vol Kolliphor (Sigma-Aldrich) and vigorous vortexing. The solution was then diluted in 87.5% vol of D5W (filtered solution of 5% dextrose in water, pH 7.0). P7C3-A20 was administered intraperitoneally (IP) daily for one month, beginning one year after TBI.

### Multimodal Traumatic Brain Injury Model

Eight week old male C57BL/6J mice were anesthetized with ketamine (100 mg/kg) and xylazine (10 mg/kg) via intraperitoneal (IP) injection and placed in an enclosed chamber constructed from an air tank partitioned into two sides and separated by a port covered by a mylar membrane. The pressure in the side not containing the mouse was increased to cause membrane rupture at 20 pounds per square inch (PSI), which generates an ˜1.0-1.5 ms air jet-flow of 137.9+/−2.09 kPa that passes through the animal's head. The head is untethered in a padded holder, while the body was fully shielded by a metal tube. The jet of air produced upon membrane rupture in this apparatus produces a collimated high-speed jet flow with extreme dynamic pressure that delivers a severe compressive impulse. Variable rupture dynamics of the diaphragm through which the jet flow is originated also generate a weak and infrequent shock front. There is also a minor component of acceleration-deceleration injury to the unrestrained head as it is agitated in the head-rest. The sham group was anesthetized and passed the same process except for the injury. The chronic study was begun with allocation of 15 animals to the Sham-Veh group, 25 animals to the TBI-Veh group, and 25 animals to the TBI-P7C3-A20 group. More animals were allocated to the TBI group in order to ensure survival of at least 15 per group by the end of the study. At the 15 month time point, relative to the day of injury, Sham-Veh group had no mortality and the TBI-Veh and TBI-P7C3-A20 groups both showed 16% mortality. At the 19 month time point, relative to the day of injury, the Sham-Veh group had no mortality, the TBI-Veh group showed 32% mortality, and the TBI-P7C3-A20 group showed 40% mortality.

### Morris Water Maze

Morris water maze was conducted in a 128 cm diameter tank filled with 19 cm of room temperature water (22° C.). White non-toxic paint was added to the water to reduce platform visibility. Four different and equally-spaced visual cues with different shapes and colors were placed inside the tank to use as reference in the location of a submerged 9 cm diameter platform. Each animal passed through a training session of four trials per day (60 sec per trial or until the mouse found the platform) for 5 consecutive days. Mice that failed to find the platform were guided to find it and allowed to stay on the platform for 30 sec. During probe test on day 6, 24 hours after the last training day, the platform was removed and each animal was monitored for 60 sec. Any-maze video tracking software (Stoelting Co.) was used to measure latency to find the hidden platform through the training session, platform crossing, distance traveled and mean speed during the probe day.

### Brain Tissue Collection

Mice were anesthetized with ketamine (100 mg/kg) and xylazine (10 mg/kg) via IP injection and transcardially perfused with phosphate-buffered saline (PBS) followed by 4% paraformaldehyde in PBS at pH 7.4. Brains were collected and postfixed in 4% paraformaldehyde in PBS at pH 7.4 overnight at 4° C., and then transferred to 30% sucrose for 72 hours. Brains were cut coronally (40 μm sections), preserved in cryoprotectant, and stored at −20° before staining.

### Electron Microscopy Analysis of BBB Integrity

Forty μm thick brain sections were washed three times with 1×PBS. Tissue was then fixed by immersion in quarter strength Karnovsky's fixative solution for 2 hours at room temperature. After washing, the specimen was postfixed for 2 hours in an unbuffered 1:1 mixture of 2% osmium tetroxide and 3% potassium ferrocyanide (Fujioka et al, 2012). After rinsing with distilled water, specimens were soaked overnight in an acidified solution of 0.25% uranyl acetate. After another rinse in distilled water, specimens were dehydrated in ascending concentrations of ethanol, passed through propylene oxide, and embedded in a EMbed 812 embedding media (Electron microscopy Sciences, Hatfield, Pa.). Thin sections (70 nm) were cut on a RMC MT6000-XL ultramicrotome. These were mounted on Gilder square 300 mesh nickel grids (Electron Microscopy Sciences, PA) and then sequentially stained with acidified methanolic uranyl acetate followed by a modification of Sato's triple lead stain (Fujioka et al., 2012). These were coated on a Denton DV-401 carbon coater (Denton Vacuum LLC, NJ), and were examined in a FEI Tecnai Spirit (T12) with a Gatan US4000 4k×4k CCD.

### REFERENCE

Fujioka H., Tandler B., Hoppel C L (2012) Mitochondrial Division in Rat Cardiomyocytes: An Electron Microscope Study. Anatomical Record. 295:1455-1461.

### Immunohistochemistry

BBB permeability was assessed by visualization of IgG in the brain. Free-floating sections (forty μm thick) were washed in PBS. Next, endogenous peroxidase activity and non-specific staining were blocked (5% BSA+5% horse serum). Sections were then incubated in biotinylated anti-mouse IgG antibody (1:500, Vector Laboratories, BA-2000) overnight at 4° C. After washing with PBS, sections were incubated with avidin-biotin complex (ABC kit, Vector laboratories, PK-4000) and developed using 3,3′-diaminobenzidine (DAB peroxidase substrate kit, Vector Laboratories, SK-4100). All sections were incubated with the same batch of DAB, and all reactions were performed at the same time and exposed for the same amount of time. Brain sections were mounted on glass slides and coverslipped with Permount. Iba1, NeuN, CD 31, and PDGFRβ staining was performed in free-floating brain sections washed in PBS and permeabilized with 0.1% Triton X-100 or 0.25% Triton X-100 (for CD 31 and PDGFRβ) in PBS. For PDGFRβ staining, antigen retrieval was performed using Dako Agilent Target Retrieval solution (S1700 RTU). Non-specific staining was blocked in 5% BSA and 5% horse serum (for Iba1 staining, endogenous peroxidase was also blocked). Brain sections were incubated in primary antibodies overnight at 4° C. After washing, sections were incubated in secondary antibodies for 1 hour at room temperature. Brain tissues were mounted on glass slides with VECTASHIELD antifade mounting medium (Vector Laboratories, H-1000, H-1200). The primary antibodies and dilutions used were as follow: anti-Iba1 (1:500, Fujifilm Wako, 019-19741); anti-NeuN (1:500, EMD Millipore Cor., #MABN-140); anti-cd 31 (1:100, BD Biosciences, 550274); and anti-PDGFRβ (1:100, R&D systems, AF1042). The secondary antibodies and dilutions used were: biotinylated anti-mouse IgG (1:1000, Vector laboratories, BA-2000); biotinylated anti-rabbit IgG (1:1000, Vector laboratories, BA-1000); anti-mouse Alexa 488 (1:1000, Invitrogen, A32723); anti-rabbit Alexa 488 (1:1000, Invitrogen, A21206); anti-goat Alexa 647(1:1000, Invitrogen, A21447). Free-floating brain sections were processed and stained with FD NeuroSilver Kit (FD NeuroTechnologies) by FD NeuroTechnologies INC (Columbia, Md., USA).

### Image Acquisition and Analysis

Images were acquired using the Zeiss Axiolmager.M2 microscope and Zeiss Axio Scan.Z1, keeping the light intensity and exposure time constant. ImageJ version 1.42 software (National Institute of Health, Bethesda, Md.) was used to analyze brightfield and fluorescent images. Silver staining (black staining) was quantified using the plugin of the color deconvolution method described by Ruifrok, A. C. & Johnston, D. A., 2001. CD 31 positive capillary length and pericytes coverage of these CD31 positive capillaries was imaged and analyzed following the method described in Bell, R. D. et. al., Neuron, 2010

### Immunoblotting

Cortical and hippocampal tissue was dissected from flash-frozen tissue and ground in RIPA buffer (Sigma-Aldrich, R0278) containing protease and phosphatase inhibitor cocktail (Thermo Scientific, #1861284). Lysates were centrifuged at 18,000 g at 4° C. for 30 min. The protein concentration of the supernatant was measured by the BCA protein assay kit (Thermo Scientific, A53225). Proteins were heated for 5 minutes in Laemmli sample buffer (Bio-Rad Laboratories, #1610737) containing beta-mercaptoethanol. Proteins were resolved in 4-20% Criterion TGX Stain-free gels (Bio-Rad Laboratories, #5678095) and transferred onto a 0.2 μm polyvinylidene fluoride membrane (Bio-Rad Laboratories, #1704157) using the Trans-Blot Turbo system (Bio-Rad Laboratories). After transfer, membranes were blocked in 5% nonfat dry milk in tris-buffered saline containing tween 20 (TBST) for 1 hour at room temperature. Then, membranes were incubated in primary antibodies overnight at 4° C. The primary antibodies and dilutions used were as follow: anti-ZO1 (1:1000, Thermo-Fischer, 33-9100); anti-occludin (1:500, BD Biosciences, B611091); anti-claudin-5 (1:1000, Thermo-Fischer, 352500); and anti-GAPDH (1:5000, EMD Millipore Corp, MAB 374). After washing in TBST, membranes were incubated with horseradish peroxidase conjugated-secondary antibodies for 1 hour and developed using SuperSignal™ West Femto Maximum Sensitivity substrate (Thermo Scientific, #34096). ImageJ version 1.42 software (National Institute of Health, Bethesda, Md.) was used to densitometry analysis.

### LPS-Induced BBB Damage Assay

BBB damage was induced by intraperitoneal injection of 3 mg/kg Lipopolysaccharides from E. coli (O111:B4, Sigma, LPS25) dissolved in saline. The damage was assessed 24 hours after LPS injection using dextran extravasation assay. The dose and duration of this assay was selected based on Banks, W. A. et. al., Journal of Neuroinflammation, 2015. For the dextran extravasation assay, we used 3 kD TMR-dextran (Invitrogen, D3308) and followed the method described in Devraj K. et. al., JOVE, 2018 with one modification-dextran was injected retro-orbitally and therefore blood/brain was collected five minutes after dextran injection. P7C3-A20 (10 mg/kg, i.p) was given 12 hours after LPS injection.

### Cytotoxicity Assay on Human Brain Microvascular Endothelial Cells

Primary human brain microvascular cells were obtained from cell system corporation, USA (Cat. No. ACBRI 376) and maintained according to manufacturer guidelines. Briefly, cells were cultured in Complete Classic Medium (Cat. No. 4Z0-500). Cells were incubated at 37° C. in 95% air and 5% C02 in a humidified incubator. Cells were grown up to 80-85% confluence and then seeded into 96 well plate (1×10⁵ cells/well) for cell viability analysis. Cells of passage number 7-11 were seeded into 96 well plate with complete serum media and grown for 24 hr before treatment. Immediately prior to treatment, medium with serum was removed and Cell Systems, Complete Serum-Free Medium (Cat No. SF-4Z0-500) with 2% FBS was added to each well. P7C3-A20 was dissolved in DMSO and further dilutions were made in the serum free medium with 2% FBS. Cells were treated with 0.1 and 0.2 mM H₂O₂, and different concentrations of P7C3 A20 (i.e., 0.03, 0.1, 0.3, 1, 1 and 5 μM). Control and H₂O₂ treated cells were also matched with vehicle concentration, i.e., 0.05% DMSO. Cells were incubated for 24 hours after treatment and cytotoxicity was measured by CyQUANT® Direct Cell Proliferation Assay Kit (Cat No. C35011), as per manufacturer protocol. Standard curve was prepared during each experiment with known amount of cells, and relative cytotoxiciy in each treatment group was calculated as the % of control cells (i.e., relative fluorescence unit in control cells with 2% FBS was considered as 100%).

### Statistical Analysis

Values are presented as mean±SEM. Statistical analyses were done using GraphPad Prism Version 8.3.0.

### REFERENCES

1. M. C. Dewan et al., Estimating the global incidence of traumatic brain injury. J Neurosurg April 1, 1-18 (2018).
2. C. A. Taylor, J. M. Bell, M. J. Breiding, L. Xu, Traumatic brain injury-related emergency department visits, hospitalizations, and deaths—United States, 2007 and 2013. MMWR Surveill Summ 66, 1-16 (2017).
3. M. Faul, C. Coronado, Epidemiology of traumatic brain injury. Handb Clin Neurol 127, 3-13 (2015).
4. V. Y. Ma, L. Chan, K. J. Carruthers, Incidence, prevalence, costs, and impact on disability of common conditions requiring rehabilitation in the United States: stroke, spinal cord injury, traumatic brain injury, multiple sclerosis, osteoarthritis, rheumatoid arthritis, limb loss, and back pain. Arch Phys Med Rehabil 95, 986-995 (2014).
5. Prevention CfDCa. Report to congress on traumatic brain injury in the United States: epidemiology and rehabilitation: Atlanta (Ga.): National Center for Injury Prevention and Control: Division of Unintentional Injury Prevention, 2015.
6. B. E. Masel, D. S. DeWitt, Traumatic brain injury: A disease process, not an event. J Neurotrauma 27, 1529-1540 (2010).
7. V. E. Johnson et al., Inflammation and white matter degeneration persist for years after a singel traumatic brain injury. Brain 136, 28-42 (2013).
8. V. E. Johnson, W. Stewart, D. H. Smith, Axonal pathology in traumatic brain injury. Exp Neurol 246, 35-43 (2013).
9. A. S. Vincent, T. M. Roebuck-Spencer, A. Cernich, Cognitive changes and dementia risk after traumatic brain injury: implications for aging military personnel. Alzheimers Dement 10(3 Supp), S174-S187 (2014).
10. R. Daneman, A Prat, The blood-brain barrier. Cold Spring Harb Perspect Biol 7: a020412 (2015).
11. R. M. Chesnut et al., A trial of intracranial-pressure monitoring in traumatic brain injury. N Engl J Med 367, 2471-2481 (2012).
12. D. O. Okonkwo et al., Brain oxygen optimization in severe traumatic brain injury phase-II: a phase II randomized Trial. Crit Care Med 45, 1907-1914 (2017).
13. P. J. D. Andrews et al., Hypothermia for intracranial hypertension after traumatic brain injury. N Engl J Med 373, 2403-2412 (2015).
14. L. Wilson et al., The chronic and evolving neurological consequences of traumatic brain injury. Lancet Neurol 16, 813-825 (2017).
15. J. D. Corrigan, F. M. Hammond, Traumatic brain injury as a chronic health condition.

Arch Phys Med Rehabil 94, 1199-1201 (2013).
16. D. S. DeWitt et al., Pre-clinical testing of therapies for traumatic brain injury. J Neurotrauma 35, 2737-2754 (2018).
17. D. K. Sandsmark, A. Bashir, C. L. Wellington, R. Diaz-Arrastia, Cerebral microvascular injury: a potentially treatable endophenotype of traumatic brain injury-induced neurodegeneration. Neuron 103, 367-379 (2019).
18. K. Kenney et al., Cerebral vascular injury in traumatic brain injury. Exp Neurol 275, 353-366 (2016).
19. O. Y. Glushakova, D. Johnson, R. L. Hayes, Delayed increases in microvascular pathology after experimental traumatic brain injury are associated with prolonged inflammation, blood-brain barrier disruption, and progressive white matter damage. J Neurotrauma 31, 1180-1193 (2014).
20. S. M. Schwarzmaier et al., In vivo temporal and spatial profile of leukocyte adhesion and migration after experimental traumatic brain injury in mice. J Neuroinflammation 10, 1-17 (2013).
21. T. C. Yin, et al., Acute axonal degeneration drives development of cognitive, motor and visual deficits after blast-mediated traumatic brain injury in mice. eNEURO 0220-16.2016 (2016).
22. E. Vázquez-Rosa et al., Neuroprotective efficacy of a sigma 2 receptor/TMEM97 modulator (DKR-1677) after traumatic brain injury. ACS Chemical Neuroscience 10, 1595-1602 (2018).
23. L. M. Dutca et al., Early detection of subclinical visual damage after blast-mediated TBI enables prevention of chronic visual deficit by treatment with P7C3-S243. Investigative Ophthalmology and Visual Sciences 55, 8330-8341 (2014).
24. M. M. Harper et al., Identification of chronic brain protein changes and protein targets of serum auto-antibodies after blast-mediated traumatic brain injury. Heliyon 6, e03374 (2020)
25. A. A. Pieper, S. L. McKnight, Evidence of benefit of enhancing nicotinamide adenine dinucleotide levels in damaged of diseased nerve cells. Cold Spring Harbor Symposium Quant Biol 83, 207-217 (2019).
26. A. A. Pieper et al., Discovery of a pro-neurogenic, neuroprotective chemical. Cell 142, 39-51. (2010).
27. A. S. Lee et al., The neuropsychiatric disease-associated gene cacna1c mediates survival of young hippocampal neurons. eNeuro, March 2016, DOI: 10.1523/ENEURO.0006-16.2016. (2016).
28. A. K. Walker et al., The P7C3 class of neuroprotective compounds blocks depressive-like behavior in ghrelin receptor—deficient mice associated with diminished posterior hippocampal neurogenesis. Mol Psych 20, 500-508 (2015).
29. M. D. Bauman et al., Neuroprotective efficacy of P7C3 compounds in primate hippocampus. Transl Psych 8, 1-11 (2018).
30. C. C. Bavely et al., Dopamine D1R-neuron cacna1c deficiency: a new model of extinction therapy-resistant post-traumatic stress. Mol Psych (2020, Apr. 24; doi: 10.1038/s41380-020-0730-8. [Epub ahead of print] PubMed PMID: 32332995.
31. K. S. MacMillan et al., Development of proneurogenic, neuroprotective small molecules. J Am Chem Soc 133, 1428-1437 (2011).
32. R. Tesla et al., Neuroprotective efficacy of aminopropyl carbazoles in a mouse model of amyotrophic lateral sclerosis. Proc Natl Acad Sci USA 109, 17016-17021 (2012).
33. H. De Jesús-Cortés et al., Neuroprotective efficacy of aminopropyl carbazoles in a mouse model Parkinson's disease. Proc Natl Acad Sci USA 109, 17010-17015 (2012).
34. J Naidoo et al., Development of a scalable synthesis of P7C3-A20, a potent neuroprotective compound. Tet Lett 54: 4429-4431 (2013).
35. M. O. Blaya, H. Bramlett, J. Naidoo, A. A. Pieper, W. D. Dietrich 3^{rd}, Neuroprotective efficacy of a proneurogenic compound after traumatic brain injury. J Neurotrauma 31, 476-486 (2014).
36. A. A. Pieper, S. L. McKnight, J. M. Ready, P7C3 and an unbiased approach to drug discovery for neurodegenerative diseases. Chem Soc Rev 43, 6716-6726 (2014).
37. S. W. Kemp et al., Pharmacologic rescue of motor and sensory function by the neuroprotective compound P7C3 following neonatal nerve injury. Neuroscience 284, 202-216 (2015).
38. J. Naidoo et al., Discovery of (−)-P7C3-S243, a Neuroprotective Aminopropyl Carbazole with Improved Drug-Like Properties. J Med Chem 57, 3746-3754 (2014).
39. T. C. Yin et al., P7C3 neuroprotective chemicals block axonal degeneration and preserve function after traumatic brain injury. Cell Reports 8, 1731-1740 (2014).
40. H. De Jesús-Cortés et al., Protective efficacy of P7C3-S243 in the 6-hydroxydopamine model of Parkinson's disease. NPJ Parkinson's Disease 1, 15010 (2015).
41. Z. B. Loris, A. A. Pieper, W. D. Dietrich 3^{rd}, The neuroprotective compound P7C3-A20 promotes neurogenesis and improves cognitive function after ischemic stroke. Exp Neurol 290, 63-73 (2017).
42. Z. B. Loris, J. R. Hynton, A. A. Pieper, W. D. Dietrich 3^{rd}, Efficacy of delayed P7C3-A20 treatment after ischemic stroke. Transl Stroke Res August 2 doi:10.1007/s12975-017-0565-z (2018).
43. J. R. Voorhees et al., P7C3 compounds protect a rat model of Alzheimer's disease from cognitive decline, depressive-like behavior, and neuronal cell death without affecting neuroinflammation or amyloid-tau pathology. Biol Psych (November 6. pii: S0006-3223(17)32145-5. doi: 10.1016/j.biopsych.2017.10.023) (2018).
44. M. O. Blaya, J. M. Wasserman, A. A. Pieper, T. J. Sick, H. M. Bramlett, W. D. Dietrich 3^{rd}, Neurotherapeutic capacity of P7C3 agents for the treatment of traumatic brain injury. Neuropharmacology 145, 268-282 (2018).
45. G. Wang et al., P7C3 neuroprotective chemicals function by activating the rate-limiting enzyme in NAD salvage. Cell 158, 1731-1740 (2014).
46. W. A. Banks et al., Lipopolysaccharide-induced blood-brain barrier disruption: roles of cyclooxygenase, oxidative stress, neuroinflammation, and elements of the neurovascular unit. J Inflammation 12, 1-15 (2015).
47. K. Devraj, S. Guerit, J. Macas, Y. Reiss, An in vivo blood-brain barrier permeability assay in mice using fluorescently labeled tracers. J Vis Exp 132, e57038 (2018).
48. B. S. Main et al., Apolipoprotein E4 impairs spontaneous blood brain barrier repair following traumatic brain injury. Mol Neurodegener 13, 17 (2018).
49. A. F. Logsdon et al., Blast exposure elicits blood-brain barrier disruption and repair mediated by tight junction integrity and nitric oxide dependent processes. Sci Rep 8, 11344 (2018).
50. P. Barzo, A. Marmarou, P. Fatouros, F. Corwin, J. Dunbar, Magnetic resonance imaging-monitored acute blood-brain barrier changes in experimental traumatic brain injury. J Neurosurg 85: 1113-1121 (1996).
51. A. Beaumont, P. Fatouros, T. Gennarelli, F. Corwin, A. Marmarou, Bolus tracer delivery measured by MRI confirms edema without blood-brain barrier permeability in diffuse traumatic brain injury. Acta Neurochir Suppl 96: 171-174 (2006).
52. S. A. Baldwin, I. Fugaccia, D. R. Brown, L. V. Brown, S. W. Scheff, Blood-brain barrier breach following cortical contusion in the rat. J. Neurosurg 85: 476-481 (1996).
53. M. K. Baskaya, A. M. Rao, A. Dogan, D. Donaldson, R. J. Dempsey, The biphasic opening of the blood-brain barrier in the cortex and hippocampus after traumatic brain injury in rats. Neurosci Lett 226: 33-36 (1997).
54. M. D. Sweeney, K. Kisler, A. Montagne, A. W. Toga, B. V. Zlokovic, The role of brain vasculature in neurodegenerative disorders. Nat Neurosci 21, 1218-1331 (2018).
55. D. Shlosberg, M. Benifla, D. Kaufer, A. Friedman, Blood-brain barrier breakdown as a therapeutic target in traumatic brain injury. Nat Rev Neurol 6, 393-403 (2010).
56. J. R. Hay, V. E. Johnson, A. M. H. Young, D. H. Smith, W. Stewart, Blood-brain barrier disruption is an early event that may persist for many years after traumatic brain injury in humans. J. Neuropathol Exp Neurol 74, 1147-1157 (2015).
57. C. P. Doherty et al., Blood-brain barrier dysfunction as a hallmark pathology in chronic traumatic encephalopathy. J Neuropathol Exp Neurol 75, 656-662 (2016).
58. E. Farkas et al., Age-related microvascular degeneration in the human cerebral periventricular white matter. Acta Neuropathol 111, 150-157 (2006).
59. D. E. Barnes et al., Association of mild traumatic brain injury with and without loss of consciousness with dementia in military veterans. JAMA Neurol 75, 1055-1061 (2018).
60. R. C. Gardner et al., Dementia risk after traumatic brain injury vs nonbrain trauma: the role of age and severity. JAMA Neurol 71, 1490-1497 (2014).
61. R. C. Garner et al., Mild TBI and risk of Parkinson disease: a chronic effects of neurotrauma consortium study. Neurology 90, e1771-e1779 (2018).
62. M. D. Sweeney, K. Kisler, A. Montagne, A. W. Toga, B. V. Zlokovic, The role of brain vasculature in neurodegenerative disorders. Nat Neurosci 21, 1318-1331 (2018).
63. P. Nordstrom, K. Michaelsson, Y. Gustafson, A. Nordstrom, Traumatic brain injury and young onset dementia: a nationwide cohort study. Ann Neurol 75, 374-381 (2014).
64. H.-K. Wang et al., Population based study on patients with traumatic brain injury suggests increased risk of dementia. J Neurol Neurosurg Psychiatry 83, 1080-1085 (2012).
65. H. T. S. Benamer, D. G. Grosset, Vascular parkinsonism: a clinical review. Eur Neurol 61, 11-15 (2009).
66. E. Bertrand et al., Amyloid angiopathy in idiopathic Parkinson's disease. Immunohistochemical and ultrastructural study. Folia Neuropathol 46, 255-270 (2008).
67. M. D. Sweeney et al., A novel sensitive assay for detection of a biomarker of pericyte injury in cerebrospinal fluid. Alz & Dementia April 16 EPub (2020).
68. D. A. Nation et al., Blood-brain barrier breakdown is an early biomarker of human cognitive dysfunction. Nat Med 25, 270-276 (2019).
69. H. J. van de Haar et al., Blood-brain barrier leakage in patients with early Alzheimer disease. Radiology 281, 527-535 (2016).
70. R. D. Bell, et al., Apolipoprotein E controls cerebrovascular integrity via cyclophilin A. Nature 485: 512-516 (2012).
71. E. H. Corder et al., Gene dose of apolipoprotein E type 4 allele and the risk of Alzheimer's disease in late onset families. Science 261: 921-923 (2003).
72. R. Mayeux et al., Synergistic effects of traumatic head injury and apolipoprotein-epsilon 4 in patients with Alzheimer's disease. Neurology 45: 555-557 (1995).

### Example 2: P7C3-A20 Treatment Increases Expression of Tight Junction Proteins in Sham and TBI Conditions

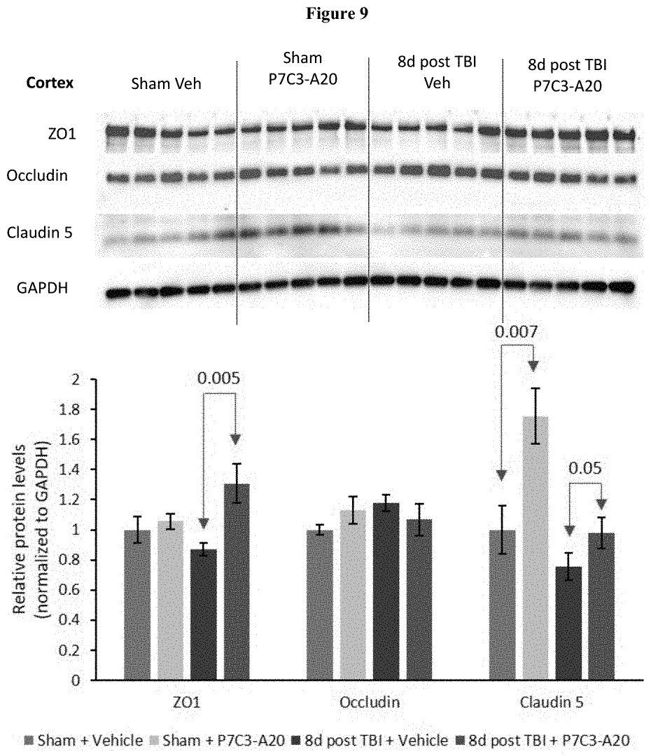

Treatment of antidepressant-resistant patients with P7C3 class of compounds can be used as a means of elevating levels of claudin 5 at the neurovascular unit in order to facilitate treatment of depression. Example 1 shows that claudin 5 is elevated by P7C3-A20 in animals that were injured. FIG. 9 also shows that claudin 5 is elevated in mice that have not been injured but are treated with P7C3-A20.

Specifically, 8-week old, male, C57BL/6J were exposed to Poly-traumatic brain injury (pTBI). The sham group was anesthetized, same as pTBI group, and passed the same process except for the injury. 24 hours following the injury, Vehicle or 10 mg/kg P7C3-A20 treatment was administered intraperitoneal twice daily for 7 days. One hour after the final injection, animals were anaesthetized and cortical tissue was collected. Protein lysates were generated from this tissue and analyzed using western blot. The primary antibodies and dilutions used were as follow: anti-ZO1 (1:1000, Thermo-Fischer, 33-9100); anti-occludin (1:500, BD Biosciences, B611091); anti-claudin-5 (1:1000, Thermo-Fischer, 352500); and anti-GAPDH (1:5000, EMD Millipore Corp, MAB 374).

1. A method of treating a disease or condition associated with insufficient levels of claudin-5, comprising:
providing a subject having insufficient levels of claudin-5, and
administering an effective amount of a P7C3 compound to the subject.

2. The method of claim 1, wherein the subject has a decreased level of claudin-5 protein compared to a healthy subject.

3. The method of claim 1, wherein the subject has a decreased level of claudin-5 mRNA compared to a healthy subject.

4. The method of claim 1, wherein the subject has a decreased level of functional claudin-5 protein compared to a healthy subject.

5. The method of claim 1, wherein the subject has a decreased level of post-translational modification of claudin-5 protein compared to a healthy subject.

6. The method of claim 5, wherein the post-translational modification comprises phosphorylation.

7. The method of claim 1, wherein the subject has damaged or deteriorated endothelial cells located in the brain, kidney, lung, heart, cornea, or digestive tissues.

8. The method of claim 7, wherein upon administration, the P7C3 compound repairs the damaged or deteriorated endothelial cells.

9. The method of claim 1, wherein the subject has a deteriorated, damaged or impaired blood-brain barrier (BBB).

10. The method of claim 9, wherein the subject shows brain permeability due to impaired BBB.

11. The method of claim 9, wherein upon administration, the P7C3 compound restores integrity, structure and/or function of the subject's BBB.

12. The method of claim 1, wherein the P7C3 compound comprises 3,6-dibromo-β-fluoro-N-(3-methoxyphenyl)-9H-carbazole-9-propanamine.

13. The method of claim 1, wherein the disease or condition is selected from Stroke, Traumatic Brain Injury, Velocardial Facial Syndrome, Epilepsy, Alzheimer's disease (AD), Glioblastoma, Multiple sclerosis, Bipolar Disorder, Obsessive Compulsive Disorder, ADHD (attention-deficit/hyperactivity disorder), Depression, Pain Disorders, Schizophrenia, and Heart Failure.

14. The method of claim 1, wherein the disease or condition is selected from subarachnoid hemorrhage, schizophrenia, major depression, bipolar disorder, normal aging, epilepsy, traumatic brain injury and/or a visual symptom associated therewith, post-traumatic stress disorder, Parkinson's disease, Alzheimer's disease, Down syndrome, spinocerebellar ataxia, amyotrophic lateral sclerosis, Huntington's disease, stroke, radiation therapy, chronic stress, abuse of a neuro-active drug, retinal degeneration, spinal cord injury, peripheral nerve injury, physiological weight loss associated with various conditions, cognitive decline and/or general frailty associated with normal aging and/or chemotherapy, chemotherapy induced neuropathy, concussive injury, crush injury, peripheral neuropathy, diabetic neuropathy, post-traumatic headache, multiple sclerosis, retinal degeneration and dystrophy (such as Leber congenital amaurosis, retinitis pigmentosa, cone-rod dystrophy, microphthalmia, anophthalmia, myopia, and hyperopia), spinal cord injury, traumatic spinal cord injury, peripheral nerve injury (such as peripheral nerve crush injury, neonatal brachial plexus palsy, and traumatic facial nerve palsy), retinal neuronal death related diseases (such as glaucoma and age related macular degeneration, diabetic retinopathy, retinal blood vessel occlusions, retinal medication toxicity (such as what amino glycosides or plaquenil can cause), retinal trauma (e.g., post-surgical), retinal infections, choroidal dystrophies, retinal pigmentary retinopathies, inflammatory and cancer mediated auto immune diseases that result in retinal neuronal cell death), Autism, Stargardt disease, Kearns-Sayre syndrome, Pure neurosensory deafness, Hereditary hearing loss with retinal diseases, Hereditary hearing loss with system atrophies of the nervous system, Progressive spinal muscular atrophy, Progressive bulbar palsy, Primary lateral sclerosis, Hereditary forms of progressive muscular atrophy and spastic paraplegia, Frontotemporal dementia, Dementia with Lewy bodies, Corticobasal degeneration, Progressive supranuclear palsy, Prion disorders causing neurodegeneration, Multiple system atrophy (olivopontocerebellar atrophy), Hereditary spastic paraparesis, Friedreich ataxia, Non-Friedreich ataxia, Spinocerebellar atrophies, Amyloidoses, Metabolic-related (e.g., Diabetes) neurodegenerative disorders, Toxin-related neurodegenerative disorders, Multiple sclerosis, Charcot Marie Tooth, Diabetic neuropathy, Metabolic neuropathies, Endocrine neuropathies, Orthostatic hypotension, Creutzfeldt-Jacob Disease, Primary progressive aphasia, Frontotemporal Lobar Degeneration, Cortical blindness, Shy-Drager Syndrome (Multiple, System Atrophy with Orthostatic Hypotension), Diffuse cerebral cortical atrophy of non-Alzheimer type, Lewy-body dementia, Pick disease (lobar atrophy), Thalamic degeneration, Mesolimbocortical dementia of non-Alzheimer type, Nonhuntingtonian types of chorea and dementia, Cortical-striatal-spinal degeneration, Dementia-Parkinson-amyotrophic lateral sclerosis complex, Cerebrocerebellar degeneration, Cortico-basal ganglionic degeneration, Familial dementia with spastic paraparesis or myoclonus, and Tourette syndrome.
